(19) United States
(12) Patent Application Publication
(10) Pub. No.: US 2024/0324967 A1
(43) Pub. Date: Oct. 3, 2024

(54) KNEE PAIN RISK ESTIMATION DEVICE, PHYSICAL CONDITION ESTIMATION SYSTEM, KNEE PAIN RISK ESTIMATION METHOD, AND RECORDING MEDIUM

(71) Applicant: NEC Corporation, Tokyo (JP)

(72) Inventors: Kazuki IHARA, Tokyo (JP); Fumiyuki NIHEY, Tokyo (JP); Chenhui HUANG, Tokyo (JP); Kenichiro FUKUSHI, Tokyo (JP); Hiroshi KAJITANI, Tokyo (JP); Yoshitaka NOZAKI, Tokyo (JP); Kentaro NAKAHARA, Tokyo (JP)

(73) Assignee: NEC Corporation, Tokyo (JP)

(21) Appl. No.: 18/601,052

(22) Filed: Mar. 11, 2024

(30) Foreign Application Priority Data
Mar. 28, 2023 (JP) 2023-051456

(51) Int. Cl. A61B 5/00; A61B 5/11

(52) U.S. Cl. CPC A61B 5/7275; A61B 5/112; A61B 5/4585; A61B 5/4824; A61B 5/6807; A61B 5/7267; A61B 5/7278; A61B 2562/0219

(57) ABSTRACT

A knee pain risk estimation device includes a feature amount construction unit that constructs a feature amount related to a sign variable to be used for estimation of a knee pain risk indicating a risk of having a medical examination in a future due to knee pain, a sign variable estimation unit that inputs the feature amount to a sign variable estimation model to estimate at least one of the sign variables, and performs a principal component analysis on the estimated at least one of the sign variables to generate a principal component vector, a knee pain risk estimation unit that estimates a knee pain risk index, and generates knee pain risk information regarding the knee pain risk by using the estimated knee pain risk index, and an output unit that outputs the generated knee pain risk information.

This application is based upon and claims the benefit of priority from Japanese Patent Application No. 2023-051456, filed on Mar. 28, 2023, the disclosure of which is incorporated herein in its entirety by reference.

### TECHNICAL FIELD

The present disclosure relates to a knee pain risk estimation device, a physical condition estimation system, a knee pain risk estimation method, and a recording medium.

### BACKGROUND ART

With growing interest in healthcare, services that provide information according to gait have attracted attention. For example, a technique for analyzing a gait using sensor data measured by a sensor mounted on footwear such as shoes has been developed. In the time-series data of the sensor data, a feature associated with a walking event related to a physical condition appears. The physical condition of the subject can be estimated by analyzing the walking data including the features associated with the walking event. For example, if the knee condition of the subject can be estimated, it is possible to detect and prevent the knee pain risk at an early stage.

PTL 1 (JP 2004-261525 A) discloses a determination device that determines knee osteoarthritis. The device of PTL 1 detects a first acceleration signal generated in a vertical direction during walking by a first acceleration sensor attached to a proximal portion of a tibia of a subject. The device of PTL 1 detects a second acceleration signal generated in the vertical direction during walking by a second acceleration sensor attached to the heel near region of the subject. The device of PTL 1 extracts a first time of the first peak point generated in the first acceleration signal. The device of PTL 1 extracts a second time of the second peak point generated in the second acceleration signal. The device of PTL 1 calculates an impact transmission period from the first time to the second time. The device of PTL 1 determines knee osteoarthritis based on whether the impact transmission period is within a predetermined range.

The device of PTL 1 determines knee osteoarthritis of the subject at the current time point by using the acceleration signals from the acceleration sensors attached to the near tibia and the near heel. However, the device of PTL 1 cannot estimate the knee pain risk that may occur in the future.

An object of the present disclosure is to provide a knee pain risk estimation device and the like capable of estimating a knee pain risk that may occur in the future.

### SUMMARY

A knee pain risk estimation device according to an aspect of the present disclosure includes a walking waveform processing unit that extracts a walking waveform for one gait cycle from sensor data measured according to a movement of a foot and normalizes the extracted walking waveform, a feature amount construction unit that constructs, by using the normalized walking waveform, a feature amount related to a sign variable to be used for estimation of a knee pain risk indicating a risk of having a medical examination in a future due to knee pain, a sign variable estimation unit that inputs the feature amount to a sign variable estimation model that outputs a sign variable to be used for estimation of the knee pain risk according to input of the feature amount to estimate at least one of the sign variables, and performs a principal component analysis on the estimated at least one of the sign variables to generate a principal component vector, a knee pain risk estimation unit that estimates a knee pain risk index by inputting the principal component vector to an index estimation model that outputs a knee pain risk index according to an input of the principal component vector, and generates knee pain risk information regarding the knee pain risk by using the estimated knee pain risk index, and an output unit that outputs the generated knee pain risk information.

A knee pain risk estimation method according to an aspect of the present disclosure causes a computer to execute extracting a walking waveform for one gait cycle from sensor data measured according to a movement of a foot, normalizing the extracted walking waveform, constructing, using the normalized walking waveform, a feature amount related to a sign variable to be used for estimation of a knee pain risk, estimating at least one of the sign variables by inputting the feature amount to a sign variable estimation model that outputs a sign variable to be used for estimation of the knee pain risk according to an input of the feature amount, generating a principal component vector by performing principal component analysis on the estimated at least one sign variable, estimating a knee pain risk index by inputting the principal component vector to an index estimation model that outputs a knee pain risk index according to an input of the principal component vector, generating knee pain risk information regarding the knee pain risk using the estimated knee pain risk index, and outputting the generated knee pain risk information.

A program according to an aspect of the present disclosure causes a computer to execute extracting a walking waveform for one gait cycle from sensor data measured according to a movement of a foot, normalizing the extracted walking waveform, constructing, using the normalized walking waveform, a feature amount related to a sign variable to be used for estimation of a knee pain risk, estimating at least one of the sign variables by inputting the feature amount to a sign variable estimation model that outputs a sign variable to be used for estimation of the knee pain risk according to an input of the feature amount, generating a principal component vector by performing principal component analysis on the estimated at least one sign variable, estimating a knee pain risk index by inputting the principal component vector to an index estimation model that outputs a knee pain risk index according to an input of the principal component vector, generating knee pain risk information regarding the knee pain risk using the estimated knee pain risk index, and outputting the generated knee pain risk information.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of a physical condition estimation system according to the present disclosure;

FIG. 2 is a block diagram illustrating an example of a configuration of a measurement device included in a physical condition estimation system according to the present disclosure;

FIG. 3 is a conceptual diagram illustrating an arrangement example of a measurement device included in a physical condition estimation system according to the present disclosure;

FIG. 4 is a conceptual diagram for explaining an example of a relationship between a local coordinate system and a world coordinate system set in a measurement device included in a physical condition estimation system according to the present disclosure;

FIG. 5 is a conceptual diagram for explaining a human body surface used in the description of the present disclosure;

FIG. 6 is a conceptual diagram for explaining a gait cycle used in the description of the present disclosure;

FIG. 7 is a block diagram illustrating an example of a configuration of a knee pain risk estimation device included in a physical condition estimation system according to the present disclosure;

FIG. 8 is a graph for explaining detection of gait parameters by a knee pain risk estimation device included in a physical condition estimation system according to the present disclosure;

FIG. 9 is a graph for explaining normalization of a walking waveform by a knee pain risk estimation device included in a physical condition estimation system according to the present disclosure;

FIG. 10 is a graph illustrating an example of time-series data of a knee joint bending angle used by a knee pain risk estimation device included in a physical condition estimation system according to the present disclosure;

FIG. 11 is a graph illustrating an example of the time-series data of an angular jerk used by a knee pain risk estimation device included in a physical condition estimation system according to the present disclosure;

FIG. 12 is a conceptual diagram for explaining estimation of a sign variable using a sign variable estimation model by a knee pain risk estimation device included in a physical condition estimation system according to the present disclosure;

FIG. 13 is a conceptual diagram for explaining principal component analysis by a knee pain risk estimation device included in a physical condition estimation system according to the present disclosure;

FIG. 14 is a conceptual diagram illustrating an estimation example of a knee pain risk index by a knee pain risk estimation device included in a physical condition estimation system according to the present disclosure;

FIG. 15 is a graph illustrating a result of verifying an example of an index estimation model used by a physical condition estimation system according to the present disclosure;

FIG. 16 is a conceptual diagram illustrating an application example of a physical condition estimation system according to the present disclosure;

FIG. 17 is a conceptual diagram illustrating an application example of a physical condition estimation system according to the present disclosure;

FIG. 18 is a flowchart for explaining an example of the operation of a measurement device included in a physical condition estimation system according to the present disclosure;

FIG. 19 is a flowchart for explaining an example of the operation of a knee pain risk estimation device included in a physical condition estimation system according to the present disclosure;

FIG. 20 is a flowchart for explaining an example of feature amount construction processing by a knee pain risk estimation device included in a physical condition estimation system according to the present disclosure;

FIG. 21 is a flowchart for explaining an example of sign variable estimation processing by a knee pain risk estimation device included in a physical condition estimation system according to the present disclosure;

FIG. 22 is a flowchart for explaining an example of knee pain risk estimation processing by a knee pain risk estimation device included in a physical condition estimation system according to the present disclosure;

FIG. 23 is a block diagram illustrating an example of a configuration of a knee pain risk estimation device according to the present disclosure;

FIG. 24 is a flowchart for explaining an example of the operation of a knee pain risk estimation device according to the present disclosure; and

FIG. 25 is a block diagram illustrating an example of a hardware configuration that executes control and processing according to the present disclosure.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

First, a physical condition estimation system according to a first example embodiment will be described with reference to the drawings. The physical condition estimation system according to the present example embodiment measures sensor data related to the movement of the foot according to the walking of the user. The physical condition estimation system according to the present example embodiment estimates an index indicating a risk of occurrence of knee pain in the future using the measured sensor data. In the present example embodiment, an example of estimating the knee pain risk is given as an index indicating the risk of the occurrence of the knee pain in the future. The knee pain risk indicates the probability of having an examination due to the pain of the knee joint within the next five years.

### Configuration

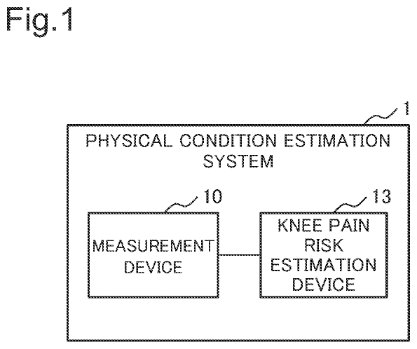

FIG. 1 is a block diagram illustrating an example of a configuration of a physical condition estimation system 1 according to the present example embodiment; The physical condition estimation system 1 includes a measurement device 10 and a knee pain risk estimation device 13. For example, the measurement device 10 is installed on footwear or the like of a subject (user) who is an estimation target of the knee pain risk. For example, the function of the knee pain risk estimation device 13 is installed in a portable terminal carried by the subject (user). Hereinafter, configurations of the measurement device 10 and the knee pain risk estimation device 13 will be individually described.

### [Measurement Device]

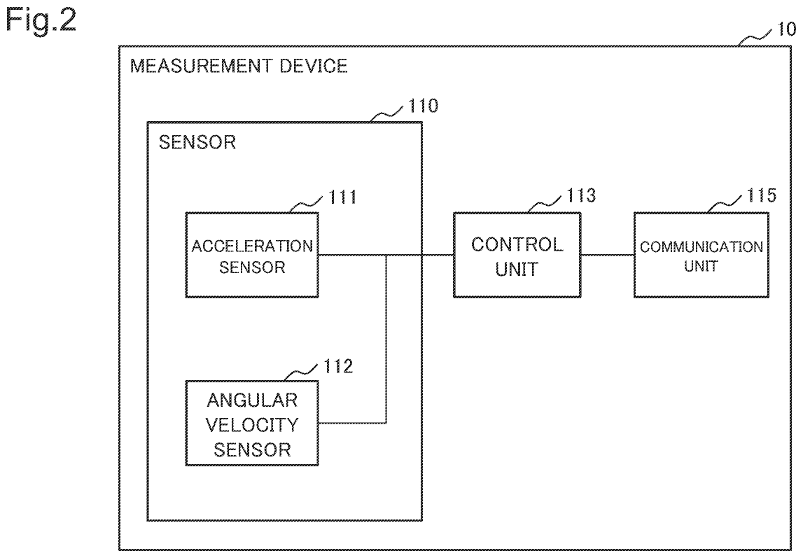

FIG. 2 is a block diagram illustrating an example of a configuration of the measurement device 10. The measurement device 10 includes a sensor 110, a control unit 113, and a communication unit 115. As illustrated in FIG. 2, the sensor 110 includes an acceleration sensor 111 and an angular velocity sensor 112. FIG. 2 illustrates an example in which the acceleration sensor 111 and the angular velocity sensor 112 are included in the sensor 110. The sensor 110 may include a sensor other than the acceleration sensor 111 and the angular velocity sensor 112. Sensors other than the acceleration sensor 111 and the angular velocity sensor 112 that can be included in the sensor 110 will not be described.

The acceleration sensor 111 is a sensor that measures accelerations (also referred to as spatial accelerations) in three axial directions. The acceleration sensor 111 measures acceleration (also referred to as spatial acceleration) as a physical quantity related to the movement of the foot. The acceleration sensor 111 outputs the measured acceleration to the control unit 113. For example, a sensor of a piezoelectric type, a piezoresistive type, a capacitance type, or the like can be used as the acceleration sensor 111. The sensor used as the acceleration sensor 111 only needs to be able to measure acceleration.

The angular velocity sensor 112 is a sensor that measures an angular velocity (also referred to as a spatial angular velocity) around three axes. The angular velocity sensor 112 measures an angular velocity (also referred to as a spatial angular velocity) as a physical quantity related to the movement of the foot. The angular velocity sensor 112 outputs the measured angular velocity to the control unit 113. For example, a sensor of a vibration type, a capacitance type, or the like can be used as the angular velocity sensor 112. The sensor used as the angular velocity sensor 112 only needs to be able to measure an angular velocity.

The sensor 110 is achieved by, for example, an inertial measurement device that measures acceleration and angular velocity. An example of the inertial measurement device is an inertial measurement unit (IMU). The IMU includes the acceleration sensor 111 that measures accelerations in three axial directions and the angular velocity sensor 112 that measures angular velocitys around three axes. The sensor 110 may be achieved by an inertial measurement device such as a vertical gyro (VG) or an attitude heading reference system (AHRS). The sensor 110 may be achieved by Global Positioning System/Inertial Navigation System (GPS/INS). The sensor 110 may be achieved by a device other than the inertial measurement device as long as it can measure a physical quantity related to the movement of the foot.

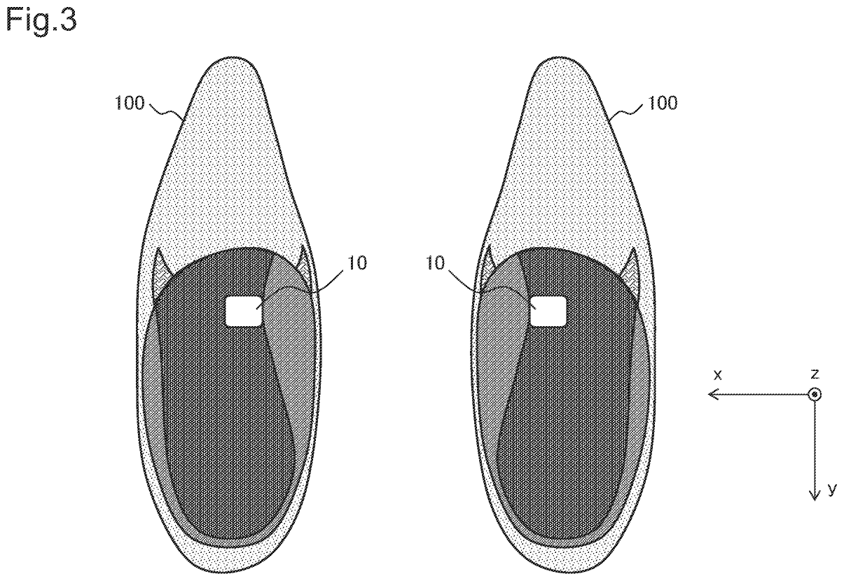

FIG. 3 is a conceptual diagram illustrating an example in which the measurement device 10 is arranged in the shoes 100 of both feet. In the example of FIG. 3, the measurement device 10 is installed at a position corresponding to the back side of the arch of foot. For example, the measurement device 10 is disposed in an insole inserted into the shoe 100. For example, the measurement device 10 may be disposed on the bottom surface of the shoe 100. For example, the measurement device 10 may be embedded in the main body of the shoe 100. The measurement device 10 may be detachable from the shoe 100 or may not be detachable from the shoe 100. The measurement device 10 may be installed at a position other than the back side of the arch of foot as long as sensor data regarding the movement of the foot can be measured. The measurement device 10 may be installed on a sock worn by the user or a decorative article such as an anklet worn by the user. The measurement device 10 may be directly attached to the foot or may be embedded in the foot.

In the example of FIG. 3, a local coordinate system including the x axis in the left-right direction, the y axis in the front-rear direction, and the z axis in the up-down direction is set with reference to the measurement device 10 (sensor 110). FIG. 3 illustrates an example in which different coordinate systems are set for the left foot and the right foot. With respect to the left foot, the x axis is positive on the left side, the y axis is positive on the front side, and the z axis is positive on the upper side. With respect to the right foot, the x axis is positive on the right side, the y axis is positive on the front side, and the z axis is positive on the upper side. The direction of the axis set in the sensor 110 may be the same for the left and right legs. For example, in a case where the sensor 110 produced with the same specification are arranged in the left and right shoes 100, the vertical orientations (orientations in the Z-axis direction) of the sensor 110 arranged in the left and right shoes 100 are the same. In this case, the three axes of the local coordinate system set to the sensor data derived from the left foot and the three axes of the local coordinate system set to the sensor data derived from the right foot are the same on the left and right.

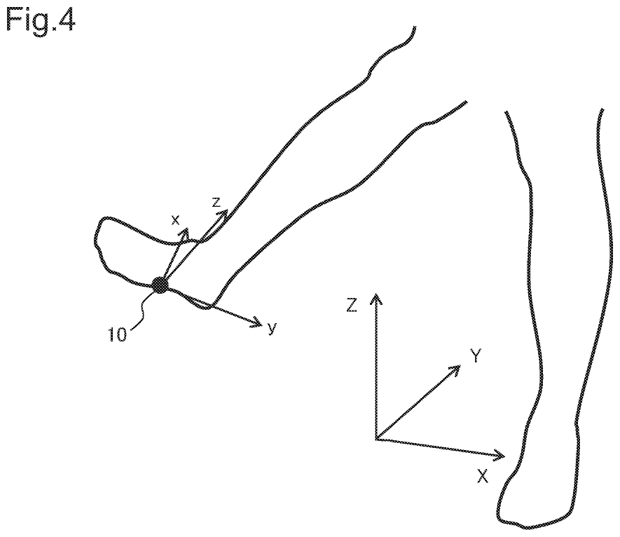

FIG. 4 is a conceptual diagram for explaining a local coordinate system (x axis, y axis, z axis) set in the measurement device 10 (sensor 110) and a world coordinate system (X axis, Y axis, Z axis) set with respect to the ground. FIG. 4 illustrates an example in which different coordinate systems are set for the left foot and the right foot. In the world coordinate system (X axis, Y axis, Z axis), in a state where the user facing the traveling direction is upright, the lateral direction of the user is set to the X-axis direction, the direction of the back surface of the user is set to the Y-axis direction, and the gravity direction is set to the Z-axis direction. The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies depending on the walking of the user.

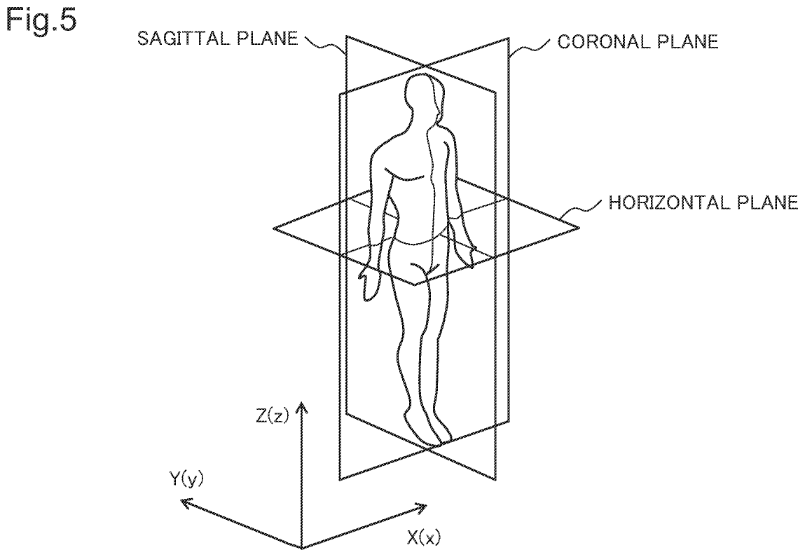

FIG. 5 is a conceptual diagram for explaining a surface (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, it is assumed that the world coordinate system and the local coordinate system coincide with each other in a state in which the center line of the foot is oriented in the traveling direction. FIG. 5 illustrates an example in which different coordinate systems are set for the left foot and the right foot. In the present example embodiment, rotation in the sagittal plane with the X axis (x axis) as the rotation axis is defined as roll, rotation in the coronal plane with the Y axis (y axis) as the rotation axis is defined as pitch, and rotation in the horizontal plane with the Z axis (z axis) as the rotation axis is defined as yaw. A rotation angle in a sagittal plane with the X axis (x axis) as a rotation axis is defined as a roll angle, a rotation angle in a coronal plane with the Y axis (y axis) as a rotation axis is defined as a pitch angle, and a rotation angle in a horizontal plane with the Z axis (z axis) as a rotation axis is defined as a yaw angle.

The control unit 113 (control means) acquires the measurement start signal transmitted from the knee pain risk estimation device 13 from the communication unit 115. The control unit 113 causes the acceleration sensor 111 and the angular velocity sensor 112 to start measurement in response to the measurement start signal. For example, the control unit 113 may cause the acceleration sensor 111 and the angular velocity sensor 112 to start measurement in response to detection of walking of the user. For example, after the heights of both feet in the vertical direction are the same over a predetermined period set in advance, the control unit 113 may be configured to start the measurement of the step width with a time point at which movement of one of the right and left feet in the traveling direction is detected as a start point. The control unit 113 may be configured to start the measurement of the step width at a predetermined timing set in advance.

The control unit 113 acquires accelerations in three axial directions from the acceleration sensor 111. The control unit 113 acquires angular velocitys around three axes from the angular velocity sensor 112. For example, the control unit 113 performs analog-to-digital conversion (AD conversion) on the physical quantities (analog data) such as measured angular velocity and acceleration. The physical quantity (analog data) measured by the acceleration sensor 111 and the angular velocity sensor 112 may be converted into digital data in each of the acceleration sensor 111 and the angular velocity sensor 112. The control unit 113 outputs the converted digital data (also referred to as sensor data) to the communication unit 115.

The control unit 113 may be configured to store the sensor data in a storage unit (not illustrated). The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition times of the data. The control unit 113 may add correction such as a mounting error, temperature correction, and linearity correction to the acceleration data and the angular velocity data.

For example, the control unit 113 is a microcomputer or a microcontroller that controls the overall measurement device 10 or processes data. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration.

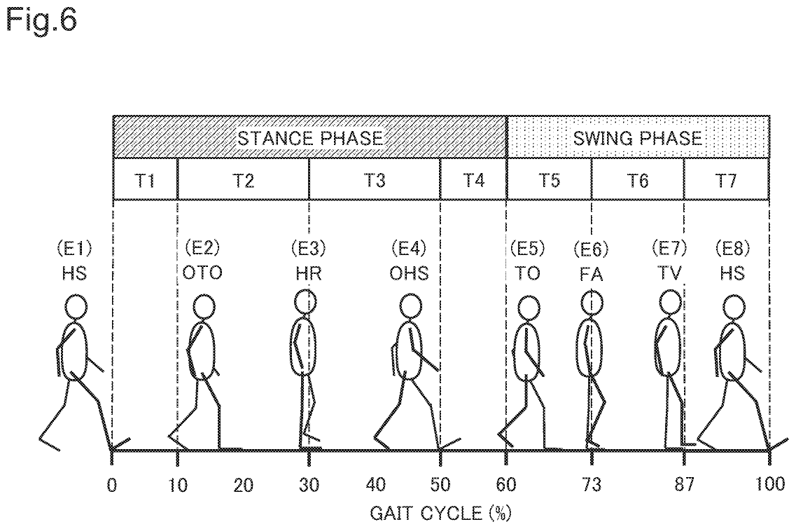

FIG. 6 is a conceptual diagram for explaining one gait cycle based on the right foot. One gait cycle based on the left foot is also similar to that of the right foot. The horizontal axis of FIG. 6 is one gait cycle of the right foot with a time point at which the heel of the right foot lands on the ground as a start point and a time point at which the heel of the right foot next lands on the ground as an end point. The horizontal axis in FIG. 6 is subjected to the first normalization with one gait cycle as 100%. In addition, the horizontal axis of FIG. 6 is subjected to the second normalization such that the stance phase is 60% and the swing phase is 40%. The one gait cycle of one foot is roughly divided into a stance phase in which at least a part of the back side of the foot is in contact with the ground and a swing phase in which the back side of the foot is separated from the ground. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting one gait cycle, the names of these periods, and the like.

As illustrated in FIG. 6, in walking, a plurality of events (also referred to as walking events) occur. E1 represents an event in which the heel of the right foot touches the ground (HS: heel strike). E2 represents an event in which the toe of the left foot is separated from the ground with the sole of the right foot in contact with the ground (OTO: opposite toe off). E3 represents an event in which the heel of the right foot lifts with the sole of the right foot in contact with the ground (HR: Heel Rise). E4 is an event in which the heel of the left foot touches the ground (OHS: Opposite Heel Strike). E5 represents an event in which the toe of the right foot is separated from the ground in a state with the sole of the left foot in contact with the ground (TO: Toe Off). E6 represents an event in which the left foot and the right foot cross with the sole of the left foot in contact with the ground (FA: Foot Adjacent). E7 represents an event that the tibia of the right foot is approximately perpendicular to the ground with the sole of the left foot in contact with the ground (TV: Tibia Vertical). E8 represents an event in which the heel of the right foot touches the ground (HS: Heel Strike). E8 corresponds to the end point of the gait cycle starting from E1 and corresponds to the start point of the next gait cycle. FIG. 6 is an example, and does not limit events that occur during walking or names of these events.

The communication unit 115 (communication means) receives the measurement start signal from the knee pain risk estimation device 13. The communication unit 115 outputs the received measurement start signal to the control unit 113. The communication unit 115 acquires sensor data measured in response to the measurement start signal from the control unit 113. The communication unit 115 transmits the acquired sensor data to the knee pain risk estimation device 13. The communication unit 115 may be configured to transmit sensor data at a preset transmission timing. For example, the communication unit 115 transmits the sensor data via wireless communication. The sensor data transmitted from the communication unit 115 is received by the knee pain risk estimation device 13. The transmission timing of the sensor data is not particularly limited. For example, the communication unit 115 transmits the sensor data in real time in response to the measurement of the sensor data. For example, the communication unit 115 may store sensor data measured during a predetermined period and collectively transmit the stored sensor data at a preset timing.

For example, the communication unit 115 transmits the sensor data to the knee pain risk estimation device 13 via wireless communication. For example, the communication unit 115 transmits the sensor data to the knee pain risk estimation device 13 via a wireless communication function (not illustrated) conforming to a standard such as Bluetooth (registered trademark) or WiFi (registered trademark). The communication function of the communication unit 115 may conform to a standard other than Bluetooth (registered trademark) or WiFi (registered trademark). The communication unit 115 may transmit the sensor data to the knee pain risk estimation device 13 via a wire such as a cable.

### [Knee Pain Risk Estimation Device]

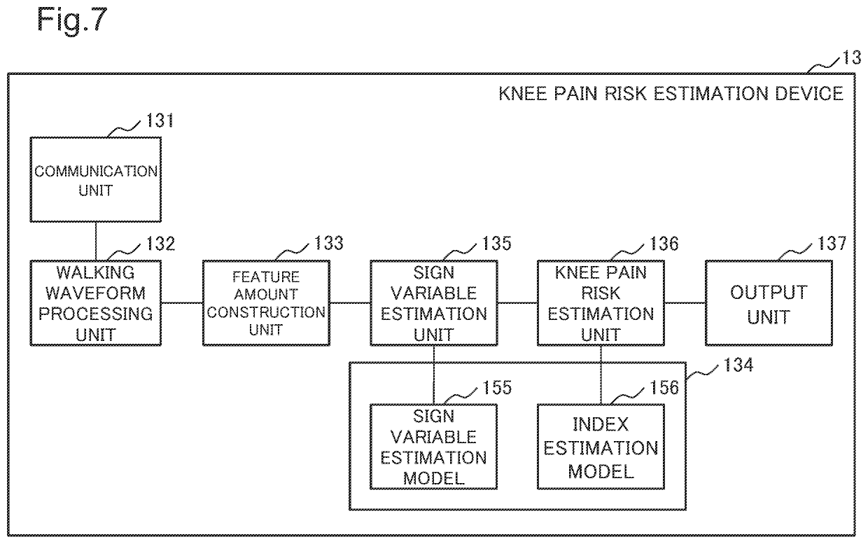

FIG. 7 is a block diagram illustrating an example of a configuration of the knee pain risk estimation device 13. The knee pain risk estimation device 13 includes a communication unit 131, a walking waveform processing unit 132, a feature amount construction unit 133, a storage unit 134, a sign variable estimation unit 135, a knee pain risk estimation unit 136, and an output unit 137. The storage unit 134 stores a sign variable estimation model 155 and an index estimation model 156. The sign variable estimation model 155 and the index estimation model 156 may be constructed in a server or a cloud connected to the knee pain risk estimation device 13 via a communication network such as the Internet.

The communication unit 131 (communication means) transmits a measurement start signal to the measurement device 10 in accordance with the measurement start timing. For example, the communication unit 131 transmits the measurement start signal at a preset time. The communication unit 131 transmits a measurement end signal to the measurement device 10 in accordance with the measurement end timing. For example, the communication unit 131 transmits the measurement end signal at a preset time. For example, the communication unit 131 transmits the measurement end signal at a stage where sensor data sufficient for generating information (knee pain risk information) according to the knee pain risk index is acquired. For example, the communication unit 131 transmits the measurement end signal at a stage where the knee pain risk information is generated. The timing of transmitting the measurement start signal and the measurement end signal may be arbitrarily set. The start and end of measurement may be controlled on the side of the measurement device 10 without transmitting the measurement start signal and the measurement end signal from the communication unit 131.

The communication unit 131 receives sensor data measured by the measurement device 10. The communication unit 131 outputs the received sensor data to the walking waveform processing unit 132. For example, the communication unit 131 receives sensor data via wireless communication. The sensor data received by the communication unit 131 is used to estimate the knee pain risk. For example, the communication unit 131 receives sensor data from the measurement device 10 via wireless communication. For example, the communication unit 131 receives the sensor data from the measurement device 10 via a wireless communication function (not illustrated) conforming to a standard such as Bluetooth (registered trademark) or WiFi (registered trademark). The communication function of the communication unit 131 may conform to a standard other than Bluetooth (registered trademark) or WiFi (registered trademark). The communication unit 131 may be configured to receive sensor data from the measurement device 10 via a wire such as a cable.

The walking waveform processing unit 132 (walking waveform processing means) acquires sensor data from the communication unit 131. The walking waveform processing unit 132 extracts time-series data (also referred to as walking waveform data) for one gait cycle from the time-series data of the acceleration in the three-axis direction and the angular velocity around the three axes included in the sensor data. The walking waveform processing unit 132 extracts walking waveform data with the timing of the heel strike HS as a start point and the timing of the next heel strike HS as an end point.

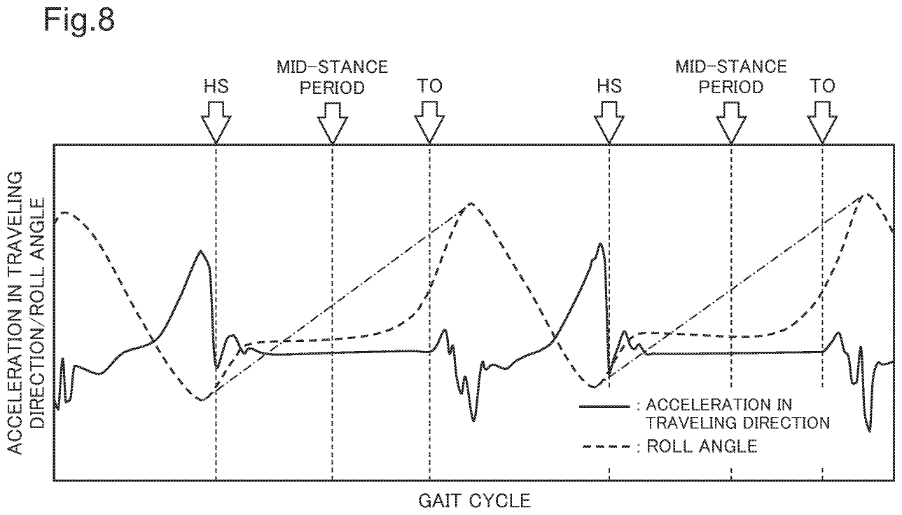

FIG. 8 is a graph for explaining an example of extracting the timing of the heel strike HS from the time-series data (solid line) of the acceleration in the traveling direction (acceleration in the Y direction). FIG. 8 also illustrates time-series data (broken line) of the roll angle (angular velocity around the X axis). FIG. 8 also illustrates the timing of the toe off TO and the mid-stance period. The timing of the heel strike HS is the timing of the local minimum peak immediately after the local maximum peak appearing in the time-series data of the acceleration in the traveling direction (acceleration in the Y direction). The local maximum peak serving as a mark of the timing of the heel strike HS corresponds to the maximum peak of the walking waveform data for one gait cycle. A section between consecutive heel strikes HS corresponds to one gait cycle. The timing of the toe off TO is the rising timing of the local maximum peak appearing after the period of the stance phase in which the fluctuation does not appear in the time-series data of the acceleration in the traveling direction (acceleration in the Y direction). The timing at the midpoint between the timing at which the roll angle is minimum and the timing at which the roll angle is maximum corresponds to the mid-stance period. For example, parameters (also referred to as gait parameters) such as walking speed, stride, minute, medial/lateral rotation, and plantarflexion/dorsiflexion can be detected with reference to the mid-stance period.

The walking waveform processing unit 132 normalizes (also referred to as first normalization) the time of the extracted walking waveform data for one gait cycle to a gait cycle of 0 to 100% (percent). Timing such as 1% or 10% included in the 0 to 100% gait cycle is also referred to as a walking phase. The walking waveform processing unit 132 normalizes (also referred to as second normalization) the first normalized walking waveform data for one gait cycle so that the stance phase becomes 60% and the swing phase becomes 40%. The stance phase is a period in which at least a part of the back side of the foot is in contact with the ground. The swing phase is a period in which the back side of the foot is away from the ground. By performing the second normalization on the walking waveform data, it is possible to reduce the shift of the walking phase from which the feature amount is extracted. The walking waveform processing unit 132 outputs the normalized walking waveform data to the feature amount construction unit 133.

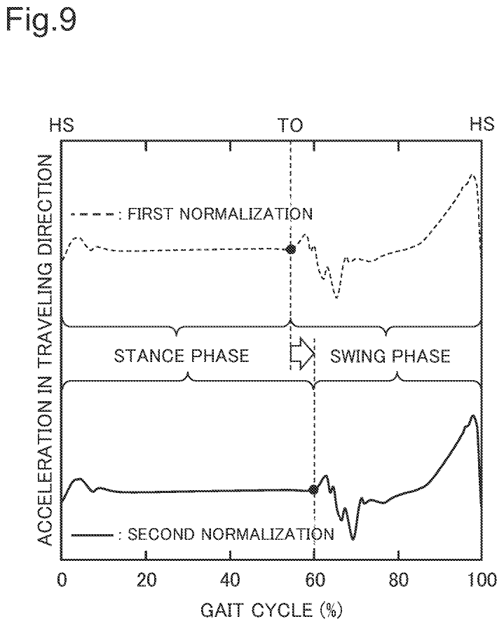

FIG. 9 is a diagram for explaining an example of walking waveform data normalized by the walking waveform processing unit 132. The walking waveform processing unit 132 detects the heel strike HS and the toe off TO from the time-series data of the acceleration in the traveling direction (acceleration in the Y direction). The walking waveform processing unit 132 extracts a section between consecutive heel strikes HS as walking waveform data for one gait cycle. The walking waveform processing unit 132 converts the horizontal axis (time axis) of the walking waveform data for one gait cycle into a gait cycle of 0 to 100% by the first normalization. In FIG. 9, the walking waveform data after the first normalization is indicated by a broken line. In the walking waveform data (broken line) after the first normalization, the timing of the toe off TO deviates from 60%.

In the example of FIG. 9, the walking waveform processing unit 132 normalizes a section from the heel strike HS with the walking phase of 0% to the toe off TO subsequent to the heel strike HS to 0 to 60%. The walking waveform processing unit 132 normalizes a section from the toe off TO to the heel strike HS in which the walking phase subsequent to the toe off TO is 100% to 60 to 100%. As a result, the walking waveform data for one gait cycle is normalized to a section (stance phase) in which the gait cycle is 0 to 60% and a section (swing phase) in which the gait cycle is 60 to 100%. In FIG. 9, the walking waveform data after the second normalization is indicated by a solid line. In the walking waveform data (solid line) after the second normalization, the timing of the toe off TO coincides with 60%.

FIGS. 8 and 9 illustrate examples in which the walking waveform data for one gait cycle is extracted/normalized based on the acceleration in the traveling direction (acceleration in the Y direction). With respect to acceleration/angular velocity other than the acceleration in the traveling direction (acceleration in the Y direction), the walking waveform processing unit 132 extracts/normalizes walking waveform data for one gait cycle in accordance with the gait cycle of the acceleration in the traveling direction (acceleration in the Y direction). The walking waveform processing unit 132 may generate time-series data of angles around three axes by integrating time-series data of angular velocitys around the three axes. In that case, the walking waveform processing unit 132 extracts/normalizes the walking waveform data for one gait cycle in accordance with the gait cycle of the acceleration in the traveling direction (acceleration in the Y direction) also for the angle around the three axes.

The walking waveform processing unit 132 may extract/normalize walking waveform data for one gait cycle based on acceleration/angular velocity other than the acceleration in the traveling direction (acceleration in the Y direction). For example, the walking waveform processing unit 132 may detect the heel strike HS and the toe off TO from the time-series data of the acceleration in the vertical direction (acceleration in the Z direction) (the drawing is omitted). The timing of the heel strike HS is a timing of a steep local minimum peak appearing in the time-series data of the acceleration in the vertical direction (acceleration in the Z direction). At the timing of the steep local minimum peak, the value of the acceleration in the vertical direction (acceleration in the Z direction) becomes substantially zero. The local minimum peak serving as a mark of the timing of the heel strike HS corresponds to the minimum peak of the walking waveform data for one gait cycle. A section between consecutive heel strikes HS is one gait cycle. The timing of the toe off TO is the timing of an inflection point in the middle of gradually increasing after the time-series data of the acceleration in the vertical direction (acceleration in the Z direction) passes through a section with a small fluctuation after the local maximum peak immediately after the heel strike HS. The walking waveform processing unit 132 may extract/normalize walking waveform data for one gait cycle based on both the acceleration in the traveling direction (acceleration in the Y direction) and the acceleration in the vertical direction (acceleration in the Z direction). The walking waveform processing unit 132 may extract/normalize the walking waveform data for one gait cycle based on acceleration, angular velocity, angle, and the like other than the acceleration in the traveling direction (acceleration in the Y direction) and the acceleration in the vertical direction (acceleration in the Z direction).

The feature amount construction unit 133 (feature amount construction means) acquires the walking waveform data from the walking waveform processing unit 132. The feature amount construction unit 133 extracts a feature amount to be used for estimation of the knee pain risk from the walking waveform data. In order to estimate the knee pain risk, the feature amount construction unit 133 constructs a feature amount to be used for estimation of a sign variable to be used for estimation of the knee pain risk. In the estimation of the knee pain risk, a sign variable related to the knee joint bending angle and a sign variable related to angular jerk cost (AJC) are used. The knee joint bending angle is an angle formed by the thigh and the lower leg around the knee joint. In the present example embodiment, the knee joint bending angle indicates an angle in a plane in the traveling direction (in the sagittal plane). The knee joint bending angle is an index of a knee disease caused by cerebral palsy, knee osteoarthritis, or the like. When such a knee disease is present, the knee joint bending angle during walking decreases. A phenomenon in which the knee joint bending angle decreases during walking in a cerebral palsy patient is called Stiff-knee Gait. AJC is a value obtained by dividing the sum of the square values of the angular jerk, which is the third derivative of the knee bending angle, by 2 and converting the sum by a logarithm having a base of 10 in a specific period included in the gait cycle. AJC is the cost of indicating the smoothness of the knee movement. AJC in a specific gait cycle is used as a sign variable.

For example, the feature amount construction unit 133 extracts the feature amount for each walking phase cluster based on a preset condition. The walking phase cluster is a cluster in which temporally continuous walking phases are integrated. The walking phase cluster includes at least one walking phase. The walking phase cluster also includes a single walking phase. The walking waveform data and the walking phase from which the feature amount used to estimate the knee pain risk is extracted will be described later. The feature amount construction unit 133 outputs the extracted feature amount for each walking phase cluster to the sign variable estimation unit 135.

For example, the feature amount construction unit 133 acquires the feature amount (first feature amount) extracted from each of the walking phases constituting the walking phase cluster. The feature amount construction unit 133 applies the feature amount constitutive expression to the acquired first feature amount to generate a feature amount (second feature amount) for each walking phase cluster. The feature amount constitutive expression is a preset calculation expression for generating the feature amount (second feature amount) for each walking phase cluster. For example, the feature amount constitutive expression is a calculation expression related to four arithmetic operations. For example, the second feature amount calculated using the feature amount constitutive expression is an integral average value, an arithmetic average value, a slope, a variation, or the like of the first feature amount in each walking phase included in the walking phase cluster. For example, the feature amount construction unit 133 applies a calculation expression for calculating the inclination and variation of the first feature amount extracted from each of the walking phases constituting the walking phase cluster as the feature amount constitutive expression. For example, in a case where the walking phase cluster is configured by a single walking phase, it is not possible to calculate the inclination or variation, and thus, it is sufficient to use a feature amount constitutive expression for calculating an integral average value, an arithmetic average value, or the like.

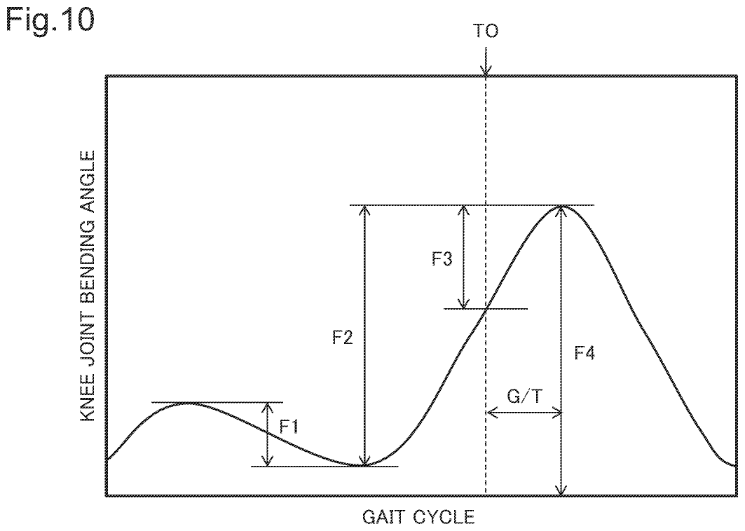

FIG. 10 is a graph illustrating an example of time-series data of a knee joint bending angle in one gait cycle. FIG. 10 is data obtained by measuring the knee flexion joint angle of the subject wearing the smart apparel by a motion capture method. The horizontal axis of FIG. 10 is the gait cycle starting from the timing of heel strike and ending at the timing of the next heel strike. The gait cycle is normalized to 0 to 100%. The gait cycle is normalized such that the stance phase is a 60% period and the swing phase is a 40% period. In the time-series data of the knee joint bending angle in one gait cycle, two peaks appear. One valley appears between the two peaks. The first peak appears in a transition period from the initial stance period T1 to the mid-stance period T2. The timing of the first peak substantially coincides with the timing of the opposite toe off OTO. The second peak appears in a transition period from the initial swing period T5 of the mid-swing period T6. The timing of the second peak substantially coincides with the timing of the foot adjacent FA.

FIG. 10 illustrates a sign variable related to the knee joint bending angle as an example of the index value indicating the knee state. In the present example embodiment, an example in which an angle variable F1, an angle variable F2, an angle variable F3, an angle variable F4, a gait cycle variable G, and a time variable T are used as the sign variable related to the knee joint bending angle will be described.

The angle variable F1 is a value obtained by subtracting the knee joint bending angle at the timing of the valley appearing between the first peak and the second peak from the knee joint bending angle at the timing of the first peak. When there is a knee disease, the valley appearing between the first peak and the second peak tends to be unclear. Therefore, when there is a knee disease, the angle variable F1 becomes small.

In order to estimate the angle variable F1, the feature amount construction unit 133 constructs feature amounts F1-1 to F1-11. The feature amount F1-1 is extracted from the walking phase 94% of the walking waveform data regarding the time-series data of the acceleration in the left-right direction (acceleration in the X direction). The feature amount F1-2 is extracted from a section of the walking phase 79 to 81% of the walking waveform data related to the time-series data of the acceleration in the traveling direction (acceleration in the Y direction). The feature amount F1-3 is extracted from the walking phases 1%, 33%, and 43% of the walking waveform data regarding the time-series data of the acceleration in the vertical direction (acceleration in the Z direction). The feature amount F1-4 is extracted from a section of the walking phase 39 to 40% of the walking waveform data related to the time-series data of the angular velocity in the coronal plane (around the Y axis). The feature amount F1-5 is extracted from a section of the walking phase 62 to 63% of the walking waveform data related to the time-series data of the angular velocity in the horizontal plane (around the Z axis). The feature amount F1-6 is extracted from a section of the walking phase 68 to 72% and 88 to 93% of the walking waveform data regarding the time-series data of the angle (posture angle) in the sagittal plane (around the X axis). The feature amount F1-7 is extracted from a section of the walking phase 6 to 21% and 23 to 28% of the walking waveform data regarding the time-series data of the angle (posture angle) in the sagittal plane (around the Y axis). The feature amount F1-8 is a stride length included in the gait parameter. The feature amount F1-9 is a maximum value (maximum dorsiflexion) of the dorsal flexion included in the gait parameter. The feature amount F1-10 is a ratio of the stance phase in one gait cycle included in the gait parameter. The feature amount F1-11 is a ratio of the swing phase in one gait cycle included in the gait parameter.

The angle variable F2 is a value obtained by subtracting the knee joint bending angle at the timing of the valley appearing between the first peak and the second peak from the knee joint bending angle at the timing of the second peak. When there is a knee disease, the valley appearing between the first peak and the second peak tends to be unclear. Therefore, when there is a knee disease, the angle variable F2 becomes small.

In order to estimate the angle variable F2, the feature amount construction unit 133 constructs feature amounts F2-1 to F2-8. The feature amount F2-1 is extracted from the walking phase 93% of the walking waveform data regarding the time-series data of the acceleration in the left-right direction (acceleration in the X direction). The feature amount F2-2 is extracted from a section of the walking phase 12% and 78 to 84% of the walking waveform data related to the time-series data of the acceleration in the traveling direction (acceleration in the Y direction). The feature amount F2-3 is extracted from the walking phases 25 to 26% of the walking waveform data regarding the time-series data of the acceleration in the vertical direction (acceleration in the Z direction). The feature amount F2-4 is extracted from a section of the walking phase 70% of the walking waveform data related to the time-series data of the angular velocity in the coronal plane (around the Y axis). The feature amount F2-5 is extracted from a section of the walking phase 38 to 44% and 63 to 86% of the walking waveform data regarding the time-series data of the angle (posture angle) in the sagittal plane (around the X axis). The feature amount F2-6 is extracted from a section of the walking phase 9 to 11% of the walking waveform data regarding the time-series data of the angle (posture angle) in the horizontal plane (around the Z axis). The feature amount F2-7 is a maximum value (maximum toe height) of the toe height included in the gait parameter. The feature amount F2-8 is a stride time included in the gait parameter.

The angle variable F3 is a value obtained by subtracting the knee joint bending angle at the timing of the toe off from the knee joint bending angle at the timing of the second peak. When there is a knee disease, the knee joint bending angle tends to decrease. Therefore, when there is a knee disease, the angle variable F3 becomes small.

In order to estimate the angle variable F3, the feature amount construction unit 133 constructs feature amounts F3-1 to F3-2. The feature amount F3-1 is extracted from the walking phases 33% and 75 to 77% of the walking waveform data regarding the time-series data of the acceleration in the vertical direction (acceleration in the Z direction). The feature amount F3-2 is extracted from a section of the walking phase 52 to 82% of the walking waveform data regarding the time-series data of the angle (posture angle) in the sagittal plane (around the X axis).

The angle variable F4 is the knee joint bending angle at the timing of the second peak. When there is a knee disease, the knee joint bending angle in the swing phase tends to decrease. Therefore, when there is a knee disease, the angle variable F4 becomes small.

In order to estimate the angle variable F4, the feature amount construction unit 133 constructs feature amounts F4-1 to F4-2. The feature amount F4-1 is extracted from the walking phase 68% of the walking waveform data regarding the time-series data of the acceleration in the left-right direction (acceleration in the X direction). The feature amount F4-2 is extracted from a section of the walking phase 75 to 86% of the walking waveform data regarding the time-series data of the angle (posture angle) in the sagittal plane (around the Y axis).

The gait cycle variable G is a temporal distance (gait cycle) from the timing of the toe off to the timing of the second peak. When there is a knee disease, the moving speed of the knee at the timing of toe off tends to decrease. Therefore, in a case where there is a knee disease, the gait cycle variable G increases.

In order to estimate the gait cycle variable G, the feature amount construction unit 133 constructs feature amounts G-1 to G-3. The feature amount G-1 is extracted from the walking phase 87% of the walking waveform data regarding the time-series data of the acceleration in the left-right direction (acceleration in the X direction). The feature amount G-2 is extracted from a section of the walking phase 76 to 78% of the walking waveform data regarding the time-series data of the angle (posture angle) in the horizontal plane (around the Z axis). The feature amount G-3 is extracted from a section of the walking phase 1 to 3% and 67 to 83% of the walking waveform data regarding the time-series data of the angle (posture angle) in the sagittal plane (around the X axis).

The time variable T is a time from the timing of the toe off to the timing of the second peak. When there is a knee disease, the moving speed of the knee at the timing of toe off tends to decrease. Therefore, when there is a knee disease, the time variable T increases.

In order to estimate the time variable T, the feature amount construction unit 133 constructs feature amounts T-1 to T-3. The feature amount T-1 is extracted from the walking phase 87% of the walking waveform data regarding the time-series data of the acceleration in the left-right direction (acceleration in the X direction). The feature amount T-2 is extracted from a section of the walking phase 76 to 78% of the walking waveform data regarding the time-series data of the angle (posture angle) in the horizontal plane (around the Z axis). The feature amount T-3 is extracted from a section of the walking phase 1 to 3% and 67 to 83% of the walking waveform data regarding the time-series data of the angle (posture angle) in the sagittal plane (around the X axis).

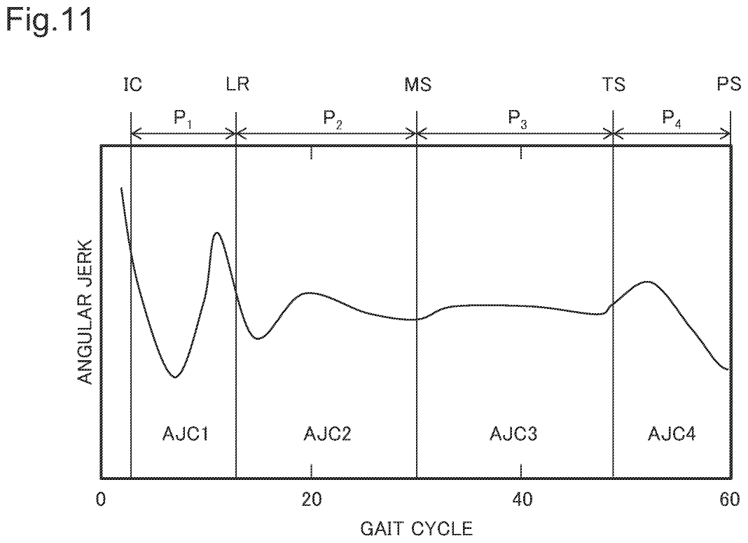

FIG. 11 is a graph illustrating an example of time-series data of angular jerk. In the present example embodiment, AJC in each of a plurality of target sections included in a section (stance phase) of 0 to 60% of one gait cycle is estimated. In the present example embodiment, AJC is estimated for each of a first section P₁, a second section P₂, a third section P₃, and a fourth section P₄ included in the stance phase. The first section P₁ is a section from an initial contact IC to a load reaction period LR. The initial contact IC is a timing immediately after the heel strike HS. The load reaction period LR is a timing when the gait cycle is about 15%. With respect to the first section P₁, AJC1 is calculated. The second section P₂ is a section from the load reaction period LR to the mid-stance MS. The mid-stance MS is the timing of transition from the mid-stance period T2 to the terminal stance period T3. The mid-stance MS is the central timing of the stance phase. With respect to the second section P₂, AJC2 is calculated. The third section P₃ is a section from the mid-stance MS to the terminal stance TS. The terminal stance TS is a timing of transition from the terminal stance period T3 to the pre-swing period T4. With respect to the third section P₃, AJC3 is calculated. The fourth section P₄ is a section from the terminal stance TS to the pre swing PS. The pre swing PS is a timing at which the toe is off. In the fourth section P₄, AJC4 is calculated.

A subject who has developed knee osteoarthritis is difficult to appropriately deal with kinematics in the initial stance period, due to function of the knee joint, walking disorder, and the like caused by factors such as knee pain and limitation of range of motion. It is estimated that such a subject responds to reduce the angular acceleration change of the knee joint by reducing the floor reaction force to ensure the smoothness of the motion to avoid knee pain. In the present example embodiment, it is assumed that the smoothness of the motion increases and the angular jerk decreases according to the compensation operation for avoiding the knee pain. Typically, the motion of the knee angle is not a constant acceleration motion. However, the motion of the knee angle tends to be close to the constant acceleration motion due to the compensation operation for alleviating the knee pain.

The storage unit 134 (storage means) stores the sign variable estimation model 155 and the index estimation model 156. The sign variable estimation model 155 outputs the sign variable related to the knee joint bending angle and the AJC according to the input of the feature amount constructed by the feature amount construction unit 133. The sign variable estimation model 155 and the index estimation model 156 may be stored in the storage unit 134 at a timing of shipment of a product from the factory, calibration before the user uses the physical condition estimation system 1, or the like. For example, the physical condition estimation system 1 may be configured to use the sign variable estimation model 155 and the index estimation model 156 stored in a storage device such as an external server. In that case, the physical condition estimation system 1 may use the sign variable estimation model 155 and the index estimation model 156 via an interface (not illustrated) connected to the storage device.

For example, the sign variable estimation model 155 and the index estimation model 156 are models constructed in advance by machine learning with an input to be described later as an explanatory variable and each estimation target as a response variable. For example, these models are constructed by learning using a linear regression algorithm. For example, these models are constructed by learning using an algorithm of a support vector machine (SVM). For example, these models are constructed by learning using a Gaussian Process Regression (GPR) algorithm. For example, these models are constructed by learning using a random forest (RF) algorithm. For example, these models may be constructed by unsupervised learning that classifies a subject who is a generation source of the feature amount data according to the feature amount data. The learning algorithm for constructing these models is not particularly limited.

The sign variable estimation model 155 and the index estimation model 156 may be stored in the storage unit 134 at a timing of shipment of a product from the factory, calibration before the user uses the physical condition estimation system 1, or the like. For example, the sign variable estimation model 155 and the index estimation model 156 may be models stored in a storage device such as an external server. In this case, the knee pain risk estimation device 13 may be configured to use these models via an interface (not illustrated) connected to the storage device.

The sign variable estimation unit 135 acquires the feature amount for each walking phase cluster from the feature amount construction unit 133. The sign variable estimation unit 135 inputs the acquired feature amount for each walking phase cluster to the sign variable estimation model 155.

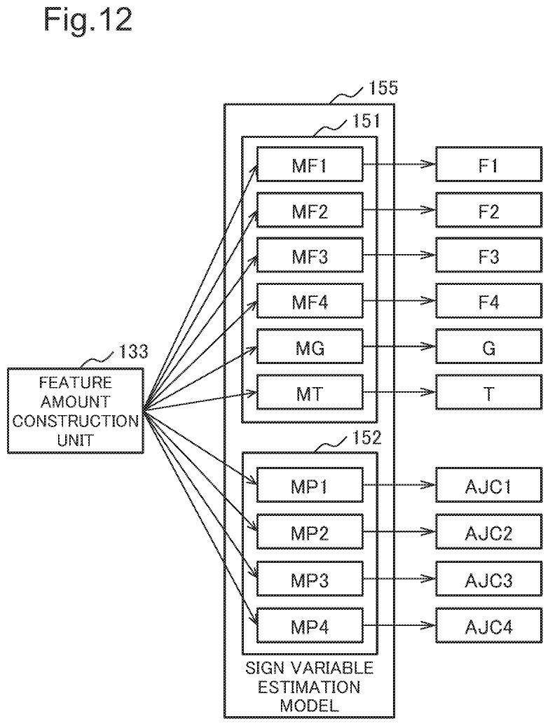

FIG. 12 is a conceptual diagram for explaining estimation of a sign variable using the sign variable estimation model 155. The sign variable estimation model 155 includes a plurality of models for estimating the sign variable. The plurality of models are divided into a first model group 151 and a second model group 152. Hereinafter, a result of verifying the correlation between the measurement value and the estimation value for a plurality of models included in the sign variable estimation model 155 will be described. In the verification, the correlation between the measurement value and the estimation value has been verified for 72 (36 males and 36 females) subjects. Specifically, a subject wearing a smart apparel has been put on a shoe on which the measurement device 10 has been mounted, and has been allowed to walk back and forth twice on a straight path of 5 m. The measurement values of the knee flexion joint angle, the gait cycle, and the time have been measured by motion capture in the walking of the subject wearing the smart apparel. AJC has been calculated using the knee flexion joint angle. The prediction value is an estimation value estimated using sensor data measured by the measurement device 10 mounted on the shoe worn by the subject at the same time as the measurement of the measurement value. Hereinafter, the correlation between the estimation value using the model and the measurement value is represented by an intraclass correlation coefficient ICC.

The first model group 151 is a model that estimates a sign variable related to a knee joint bending angle. The first model group 151 includes a first angle variable estimation model MF1, a second angle variable estimation model MF2, a third angle variable estimation model MF3, and a fourth angle variable estimation model MF4. The first model group 151 includes a gait cycle variable estimation model MG and a time variable estimation model MT.

The first angle variable estimation model MF1 is a model that outputs the angle variable F1 according to the input of the feature amount. For example, the first angle variable estimation model MF1 outputs the angle variable F1 according to inputs of the feature amounts F1-1 to F1-11. The first angle variable estimation model MF1 is learned using training data having the feature amounts F1-1 to F1-11 to be used for estimation of the angle variable F1 as explanatory variables and the angle variable F1 as an objective variable. In the verification described above, the intraclass correlation coefficient ICC between the estimation value using the first angle variable estimation model MF1 and the measurement value of the angle variable F1 has been 0.4893.

The second angle variable estimation model MF2 is a model that outputs the angle variable F2 according to the input of the feature amount. For example, the second angle variable estimation model MF2 outputs the angle variable F2 according to inputs of the feature amounts F2-1 to F2-8. The second angle variable estimation model MF2 is learned using training data having the feature amounts F2-1 to F2-8 to be used for estimation of the angle variable F2 as explanatory variables and the angle variable F2 as an objective variable. In the verification described above, the intraclass correlation coefficient ICC between the estimation value using the second angle variable estimation model MF2 and the measurement value of the angle variable F2 has been 0.4732.

The third angle variable estimation model MF3 is a model that outputs the angle variable F3 according to the input of the feature amount. For example, the third angle variable estimation model MF3 outputs the angle variable F3 according to inputs of the feature amounts F3-1 to F3-2. The third angle variable estimation model MF3 is learned using training data having the feature amounts F3-1 to F3-2 to be used for estimation of the angle variable F3 as explanatory variables and the angle variable F3 as an objective variable. In the verification described above, the intraclass correlation coefficient ICC between the estimation value using the third angle variable estimation model MF3 and the measurement value of the angle variable F3 has been 0.5944.

The fourth angle variable estimation model MF4 is a model that outputs the angle variable F4 according to the input of the feature amount. For example, the fourth angle variable estimation model MF4 outputs the angle variable F4 according to inputs of the feature amounts F4-1 to F4-2. The fourth angle variable estimation model MF4 is learned using training data having the feature amounts F4-1 to F4-2 to be used for estimation of the angle variable F4 as explanatory variables and the angle variable F4 as an objective variable. In the verification described above, the intraclass correlation coefficient ICC between the estimation value using the fourth angle variable estimation model MF4 and the measurement value of the angle variable F4 has been 0.3345.

The gait cycle variable estimation model MG is a model that outputs the gait cycle variable G according to the input of the feature amount. For example, the gait cycle variable estimation model MG outputs the gait cycle variable G according to the input of the feature amounts G-1 to G-3. The gait cycle variable estimation model MG is learned using training data having the feature amounts G-1 to G-3 to be used for estimation of the gait cycle variable G as explanatory variables and the gait cycle variable G as an objective variable. In the verification described above, the intraclass correlation coefficient ICC between the estimation value using the gait cycle variable estimation model MG and the measurement value of the gait cycle variable G has been 0.4818.

The time variable estimation model MT is a model that outputs the time variable T according to the input of the feature amount. For example, the time variable estimation model MT outputs the time variable T according to the input of the feature amounts T-1 to T-3. The time variable estimation model MT is learned using training data having the feature amounts T-1 to T-3 to be used for estimation of the time variable T as explanatory variables and the time variable T as an objective variable. In the verification described above, the intraclass correlation coefficient ICC between the estimation value using the time variable estimation model MT and the measurement value of the time variable T has been 0.7122.

The second model group 152 is a model for estimating a sign variable related to AJC. The second model group 152 includes an AJC estimation model MP1, an AJC estimation model MP2, an AJC estimation model MP3, and an AJC estimation model MP4.

The AJC estimation model MP1 is a model that outputs the AJC1 according to the input of the feature amount. For example, the AJC estimation model MP1 is learned by using training data in which at least one feature amount to be used for estimation of the AJC estimation model MP1 is an explanatory variable and the AJC estimation model MP1 is an objective variable. In the above verification, the intraclass correlation coefficient ICC between the estimation value using the AJC estimation model MIPl and the measurement value of AJC1 has been 0.2453.

The AJC estimation model MP2 is a model that outputs the AJC2 according to the input of the feature amount. For example, the AJC estimation model MP2 is learned by using training data in which at least one feature amount to be used for estimation of the AJC estimation model MP2 is an explanatory variable and the AJC estimation model MP2 is an objective variable. In the above verification, the intraclass correlation coefficient ICC between the estimation value using the AJC estimation model MP2 and the measurement value of AJC2 has been 0.4418.

The AJC estimation model MP3 is a model that outputs the AJC3 according to the input of the feature amount. For example, the AJC estimation model MP3 is learned by using training data having at least one feature amount to be used for estimation of the AJC estimation model MP3 as an explanatory variable and the AJC estimation model MP3 as an objective variable. In the above verification, the intraclass correlation coefficient ICC between the estimation value using the AJC estimation model MP3 and the measurement value of AJC3 has been 0.6114.

The AJC estimation model MP4 is a model that outputs the AJC4 according to the input of the feature amount. For example, the AJC estimation model MP4 is learned by using training data in which at least one feature amount to be used for estimation of the AJC estimation model MP4 is an explanatory variable and the AJC estimation model MP4 is an objective variable. In the above verification, the intraclass correlation coefficient ICC between the estimation value using the AJC estimation model MP4 and the measurement value of AJC4 has been 0.6185.

The intraclass correlation coefficient ICC between the measurement value and the estimation value has been different depending on the section. In the first section P₁, the movement of the measurement device 10 is complicated, and noise is likely to be included in the sensor data. As a result, it is estimated that the measurement value, the estimation value, and the intraclass correlation coefficient ICC have decreased. On the other hand, in the third section P₃ and the fourth section P₄, the movement of the measurement device 10 is stabilized, and it is estimated that the measurement value, the estimation value, and the intraclass correlation coefficient ICC are relatively good.

The sign variable estimation unit 135 (sign variable estimation means) performs the principal component analysis on the plurality of sign variables output from the sign variable estimation model 155. That is, the sign variable estimation unit 135 performs the principal component analysis on the outputs of the first model group 151 and the second model group 152. For example, the sign variable estimation unit 135 performs the principal component analysis on the plurality of sign variables using a principal component calculation expression constructed in advance based on the learning data. For example, the sign variable estimation unit 135 may perform the principal component analysis on a plurality of sign variables using a principal component analysis model (not illustrated) learned in advance. The principal component analysis model performs principal component analysis PCA according to the input of the sign variable. The sign variable estimation model 155 outputs a principal component vector PCV including at least one principal component.

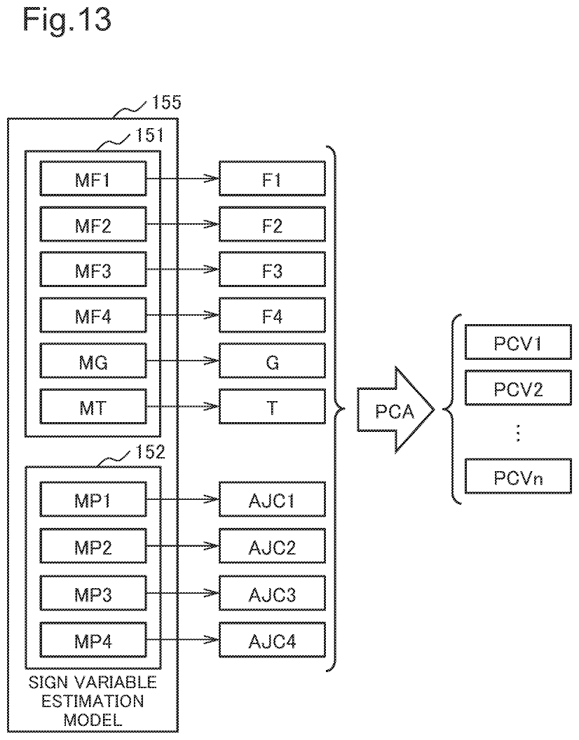

FIG. 13 is a conceptual diagram for explaining the principal component analysis PCA by the sign variable estimation unit 135. Specifically, the sign variable estimation unit 135 executes the principal component analysis using the angle variable F1, the angle variable F2, the angle variable F3, the angle variable F4, the gait cycle variable G, the time variable T, AJC1, AJC2, AJC3, and AJC4. The sign variable estimation unit 135 generates a principal component vector PCV1, a principal component vector PCV2, . . . , and a principal component vector PCVn by the principal component analysis (n is a natural number).

For example, the sign variable estimation unit 135 constructs a principal component vector PSV by performing the principal component analysis on the sign variables related to a plurality of subjects classified into two groups depending on the presence or absence of knee pain. For example, the sign variable estimation unit 135 calculates d of Cohen, which is an index for quantitatively evaluating the degree of separation of the distribution between the two groups. d of Cohen is a value (amount of effect) obtained by dividing the difference between the average values between the two samples by the standard deviation and standardizing the value. d of Cohen represents how far the average values of the two samples is apart. The larger the value of d of Cohen is, the more the average values in the two specimens are apart from each other, which is effective in estimating the knee pain risk. The value calculated by the sign variable estimation unit 135 is not limited to d of Cohen. For example, the sign variable estimation unit 135 may calculate g of Hedges.

The knee pain risk estimation unit 136 (knee pain risk estimation means) acquires the principal component vector PSV constructed by the sign variable estimation unit 135. The knee pain risk estimation unit 136 inputs the acquired principal component vector PSV to the index estimation model 156 to estimate a knee pain risk index.

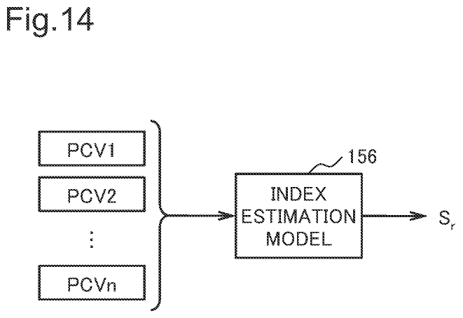

FIG. 14 is a conceptual diagram illustrating an estimation example of a knee pain risk index Sᵣ by the knee pain risk estimation unit 136. The index estimation model 156 is a model generated by learning using training data having the principal component vector PSV as an explanatory variable and the knee pain risk index Sᵣ as an objective variable. The knee pain risk index Sᵣ is an index indicating the possibility of having a medical examination due to knee pain in the future. For example, the knee pain risk index Sᵣ indicates the possibility of having a medical examination due to knee pain after five years. For example, the knee pain risk index Sᵣ is a score expressed numerically. For example, the knee pain risk index Sᵣ may be a determination such as no knee pain risk (determination A), a possibility of having a medical examination in five years (determination B), a reliable medical examination in five years (determination C), or an already medical examination (determination D). If the knee pain risk index Sᵣ is an index indicating the possibility of having a medical examination due to knee pain in the future, the knee pain risk index Sᵣ may be a determination reference that does not indicate the possibility of having a medical examination due to knee pain after five years.

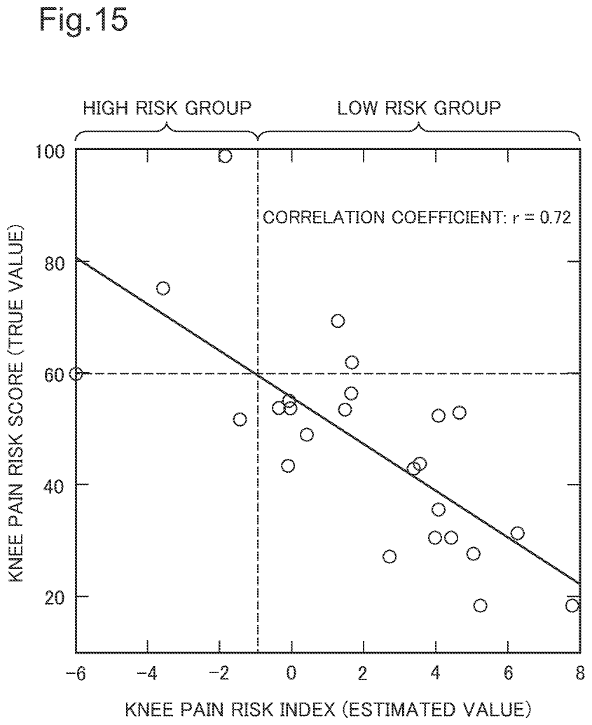

FIG. 15 is a graph illustrating a result of verifying the index estimation model 156. FIG. 15 illustrates a correlation between a knee pain risk index (estimation value) estimated using the index estimation model 156 and a knee pain risk score (true value) evaluated by an expert regarding walking of a plurality of subjects. Here, a verification example performed on 25 (6 males and 19 females) subjects will be described. In this verification, the subject wearing the shoe on which the measurement device 10 is mounted has been caused to walk 4 times on a straight path of 10 m at a normal walking speed. The walking state of the subject has been recorded by taking moving images from the side, the front, and the back. The knee pain risk index of the subject has been determined by a specialist panel using a moving image obtained by photographing the walking state of the subject. The specialist panel includes doctors and physical therapists. In the estimation of the knee pain risk index using the index estimation model 156, a principal component vector obtained by performing the principal component analysis on the above-described 10 sign variables has been used.

FIG. 15 illustrates a regression line indicating a correlation between the knee pain risk index (estimation value) estimated using the index estimation model 156 and the knee pain risk score (true value) evaluated by an expert. The correlation coefficient r of the regression line has been 0.72. The determination threshold of the knee pain risk based on the knee pain risk score (true value) is 60 points. Regarding the knee pain risk score (true value), 60 points or more are in a high risk group, and less than 60 points are in a low risk group. In the example of FIG. 15, the knee pain risk index (estimation value) corresponding to 60 points which is the determination threshold of the knee pain risk score (true value) is −1. In the example of FIG. 15, with respect to the knee pain risk index (estimation value), the high risk group is −1 or less, and the low risk group is more than −1. That is, a subject (user) having a knee pain risk index (estimation value) of −1 or less has a knee pain risk.

The knee pain risk estimation unit 136 generates information (knee pain risk information) corresponding to the knee pain risk index (estimation value) estimated using the index estimation model 156. For example, the knee pain risk estimation unit 136 generates information including an estimation value of the knee pain risk index as the knee pain risk information. For example, the knee pain risk estimation unit 136 generates information including an estimation result corresponding to the estimation value of the knee pain risk index as the knee pain risk information. For example, the knee pain risk estimation unit 136 generates information including action recommendation corresponding to the estimation value of the knee pain risk index as the knee pain risk information. The knee pain risk information generated by the knee pain risk estimation unit 136 is not particularly limited as long as it is information regarding the knee pain risk.

The output unit 137 (output means) acquires knee pain risk information estimated by the knee pain risk estimation unit 136. The output unit 137 outputs the acquired knee pain risk information. For example, the output unit 137 displays the knee pain risk information on the screen of a portable terminal 180 of the subject (user). For example, the output unit 137 outputs the knee pain risk information to an external system or the like that uses the knee pain risk information. The use of the knee pain risk information output from the output unit 137 is not particularly limited.

Here, a display example of the knee pain risk information output from the knee pain risk estimation device 13 will be described with reference to the drawings. The following display example illustrates an example in which the function of the knee pain risk estimation device 13 installed in the portable terminal carried by the user estimates the knee pain risk information using the sensor data measured by the measurement device 10 mounted on the shoe.

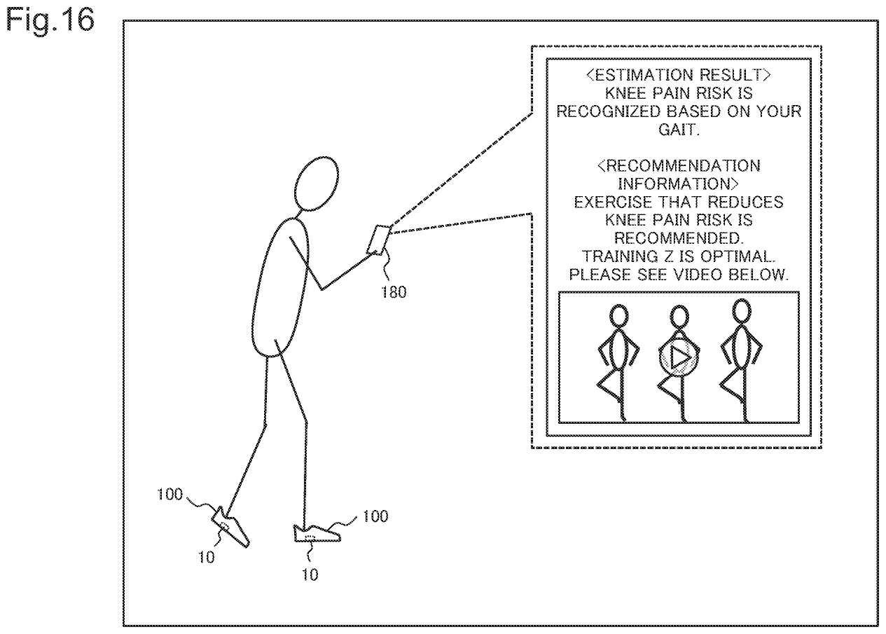

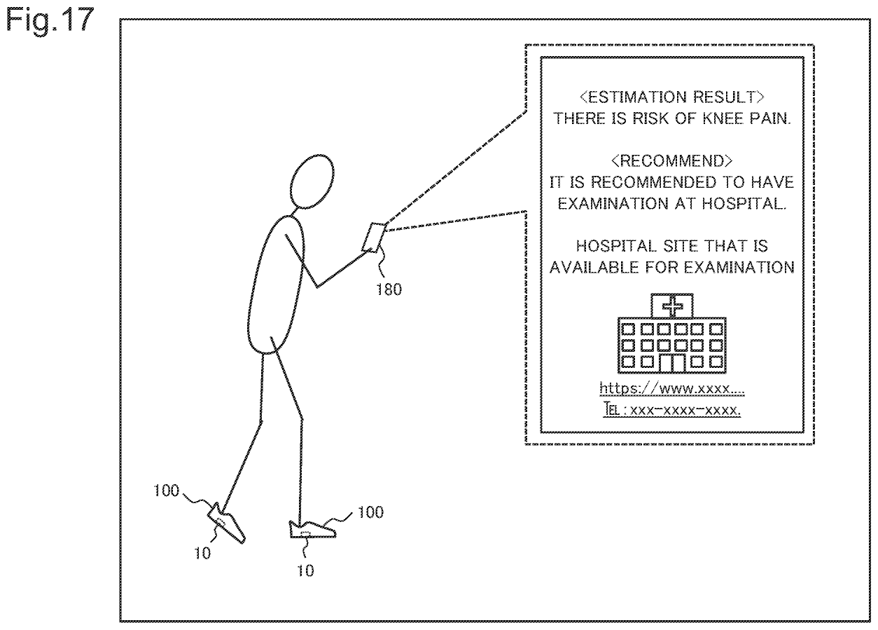

FIGS. 16 and 17 are conceptual diagrams illustrating an example of displaying the estimation result by the knee pain risk estimation device 13 on the screen of the portable terminal 180 carried by the user walking while wearing the shoe 100 on which the measurement device 10 is disposed. FIGS. 16 and 17 are examples in which the knee pain risk information according to the estimation result of the knee pain risk using the feature amount data based on the sensor data measured according to the walking of the user is displayed on the screen of the portable terminal 180.

FIG. 16 illustrates an example in which the knee pain risk information corresponding to the estimation result of the knee pain risk is displayed on the screen of the portable terminal 180. In the example of FIG. 16, the knee pain risk information “Knee pain risk is recognized based on your gait.” is displayed on the screen of the portable terminal 180 according to the estimation result of the knee pain risk. In the example of FIG. 16, in accordance with the estimation result of the knee pain risk, recommendation information according to the estimation result of the knee pain risk such as “Exercise that reduces the knee pain risk is recommended. The training Z is optimal. Please see the video below.” is displayed on the display unit of the portable terminal 180. The user who has confirmed the information displayed on the display unit of the portable terminal 180 can practice training leading to avoidance of the knee pain risk by exercising with reference to the video of the training Z according to the recommendation information.

FIG. 17 illustrates another example in which the knee pain risk information corresponding to the estimation result of the knee pain risk is displayed on the screen of the portable terminal 180. In the example of FIG. 16, the knee pain risk information “There is a risk of knee pain.” is displayed on the screen of the portable terminal 180 according to the estimation result of the knee pain risk. In the example of FIG. 16, the recommendation information “It is recommended to have an examination at a hospital.” is displayed on the display unit of the portable terminal 180 according to the estimation result of the knee pain risk. For example, a link destination or a telephone number to a hospital site that is available for examination may be displayed on the screen of the portable terminal 180. The user who has confirmed the information displayed on the display unit of the portable terminal 180 can appropriately receive an examination of a disease related to the knee by going to a hospital according to the recommendation information.

The estimated knee pain risk information may be provided to a person other than the user. For example, the knee pain risk information may be output to a terminal device (not illustrated) used by a trainer, a doctor, a family member of the user, or the like that manages the physical condition of the user. For example, the knee pain risk information may be recorded in a database (not illustrated) constructed for the purpose of health management or the like.

### Operation

Next, an example of an operation of the physical condition estimation system 1 will be described with reference to the drawings. Here, the measurement device 10 and the knee pain risk estimation device 13 included in the physical condition estimation system 1 will be individually described. With regard to the measurement device 10, the operation of the components included in the measurement device 10 will be described.

### [Measurement Device]

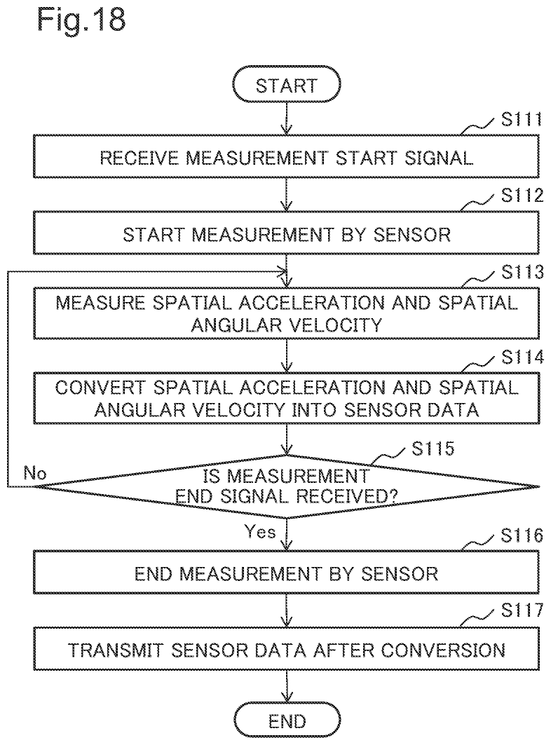

FIG. 18 is a flowchart for explaining the operation of the measurement device 10. In the description regarding the processing along the flowchart of FIG. 18, the components of the measurement device 10 will be described as the operation subject.

In FIG. 18, first, the communication unit 115 receives the measurement start signal (step S111). The measurement start signal is a signal transmitted from the knee pain risk estimation device 13.

Next, the control unit 113 starts measurement by the sensor 110 in response to the measurement start signal (step S112).

Next, the sensor 110 measures the spatial acceleration and the spatial angular velocity (step S113). The spatial acceleration is measured by the acceleration sensor 111. The spatial angular velocity is measured by the angular velocity sensor 112.

Next, the control unit 113 converts the spatial acceleration and the spatial angular velocity into sensor data (step S114).

Here, when the communication unit 115 receives the measurement end signal (Yes in step S115), the control unit 113 ends the measurement by the sensor 110 (step S116). At a stage where the communication unit 115 has not received the measurement end signal (No in step S115), the control unit 113 continues the measurement by the sensor 110 (returns to step S113).

After step S116, the communication unit 115 transmits the converted sensor data (step S117). The transmitted sensor data is time-series data. The time-series data of the sensor data is used for estimation of knee pain risk information by the knee pain risk estimation device 13.

### [Knee Pain Risk Estimation Device]

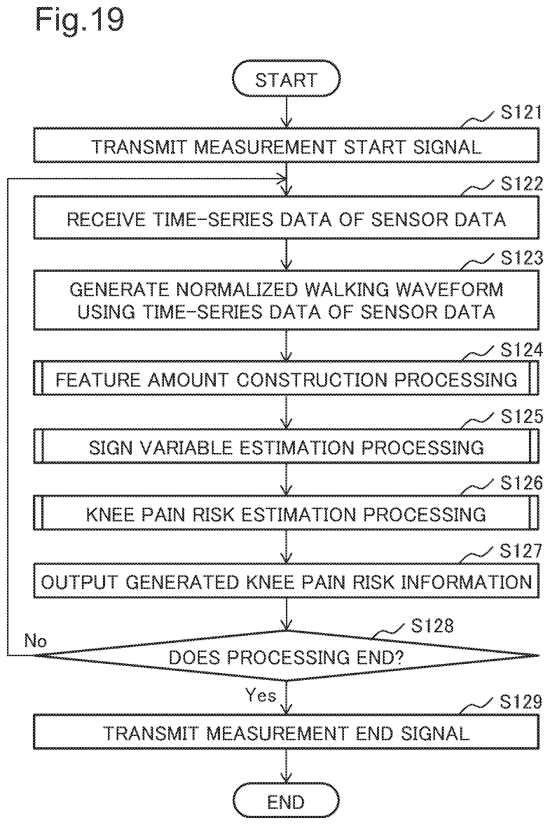

FIG. 19 is a flowchart for explaining an operation of the knee pain risk estimation device 13. In the description along the flowchart of FIG. 19, the components of the knee pain risk estimation device 13 will be described as an operation subject.

In FIG. 19, first, the communication unit 131 transmits a measurement start signal (step S121). The measurement start signal is received by the measurement device 10. When the measurement device 10 controls the start of measurement, step S121 is omitted.

Next, the communication unit 131 receives the time-series data of the sensor data (step S122).

Next, the walking waveform processing unit 132 generates a normalized walking waveform using the time-series data of the sensor data (step S123). In step S123, the walking waveform processing unit 132 detects the heel strike and the toe off from the time-series data of the sensor data. The walking waveform processing unit 132 extracts time-series data of a section between consecutive heel strikes as walking waveform data for one gait cycle. The walking waveform processing unit 132 normalizes the walking waveform data for one gait cycle to a gait cycle of 0 to 100% (first normalization). Furthermore, the walking waveform processing unit 132 normalizes the ratio of the stance phase to the swing phase in the walking waveform data subjected to of the first normalization for one gait cycle to 60:40 (second normalization).

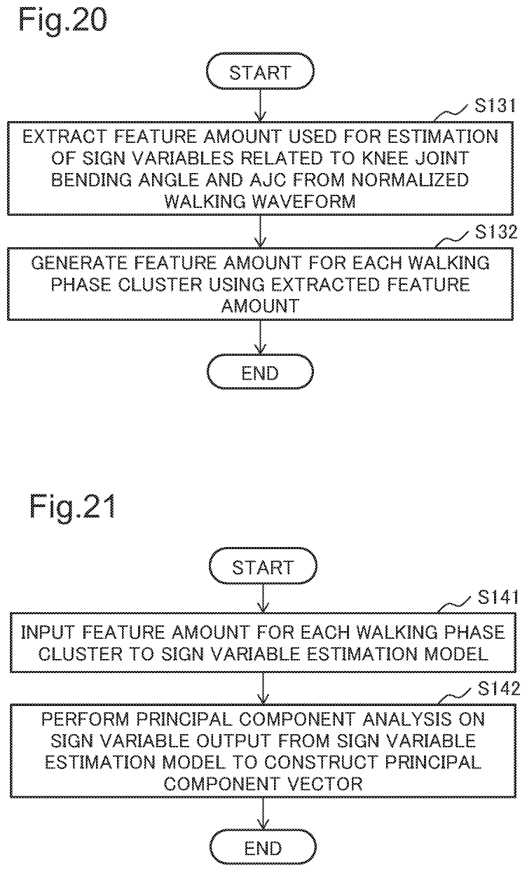

Next, the feature amount construction unit 133 executes feature amount construction processing (step S124). Details of the feature amount construction processing will be described later (FIG. 20).

Next, the sign variable estimation unit 135 executes sign variable construction processing (step S125). Details of the feature amount construction processing will be described later (FIG. 21).

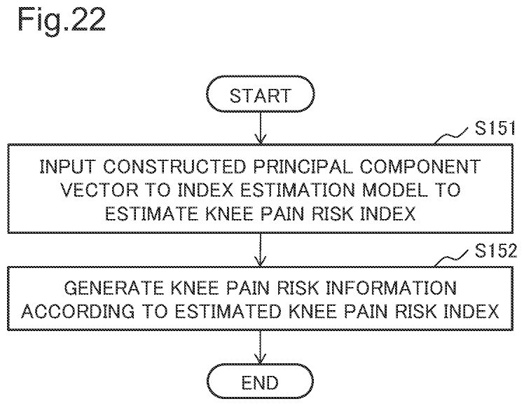

Next, the knee pain risk estimation unit 136 executes knee pain risk estimation processing (step S126). Details of the feature amount construction processing will be described later (FIG. 22).

Next, the output unit 137 outputs the generated knee pain risk information (step S127).

When ending the processing (Yes in step S128), the communication unit 131 transmits a measurement end signal (step S129). The measurement end signal is received by the measurement device 10. When the process is continued (No in step S128), the process returns to step S122. When the measurement device 10 controls the measurement end, step S129 is omitted.

### <Feature Amount Construction Processing>

FIG. 20 is a flowchart for explaining the feature amount construction processing (step S124 in FIG. 19). In the description along the flowchart of FIG. 20, the feature amount construction unit 133 will be described as an operation subject.

In FIG. 20, first, the feature amount construction unit 133 extracts a feature amount to be used for estimation of a knee joint bending angle and a sign variable related to AJC from the normalized walking waveform (step S131).

Next, the feature amount construction unit 133 generates a feature amount for each walking phase cluster using the extracted feature amount (step S132). After step S132, the process proceeds to sign variable construction processing (FIG. 21) in step S125 of FIG. 19.

### <Sign Variable Construction Processing>

FIG. 21 is a flowchart for explaining the sign variable construction processing (step S125 in FIG. 19). In the description along the flowchart of FIG. 21, the sign variable estimation unit 135 will be described as an operation subject.

In FIG. 21, first, the sign variable estimation unit 135 inputs the feature amount for each walking phase cluster to the sign variable estimation model 155 (step S141).

Next, the sign variable estimation unit 135 performs the principal component analysis on the sign variable output from the sign variable estimation model 155 to construct a principal component vector (step S142). After step S142, the process proceeds to sign variable estimation processing (FIG. 22) in step S126 of FIG. 19.

### <Knee Pain Risk Estimation Processing>

FIG. 22 is a flowchart for explaining the knee pain risk estimation processing (step S126 in FIG. 19). In the description along the flowchart of FIG. 22, the knee pain risk estimation unit 136 will be described as an operation subject.

In FIG. 22, first, the knee pain risk estimation unit 136 inputs the constructed principal component vector to the index estimation model 156 to estimate a knee pain risk index (step S151).

Next, the knee pain risk estimation unit 136 generates knee pain risk information according to the estimated knee pain risk index output from the index estimation model 156 (step S152). After step S152, the process proceeds to step S127 in FIG. 19.

As described above, the physical condition estimation system of the present example embodiment includes the measurement device and the knee pain risk estimation device. The measurement device is installed on the footwear of the user to whom the knee pain risk information is to be estimated. The measurement device measures a spatial acceleration and a spatial angular velocity. The measurement device generates sensor data corresponding to walking using the measured spatial acceleration and spatial angular velocity. The measurement device transmits the generated sensor data to the knee pain risk estimation device.

The knee pain risk estimation device includes a communication unit, a walking waveform processing unit, a feature amount construction unit, a storage unit, a sign variable estimation unit, a knee pain risk estimation unit, and an output unit. The communication unit acquires sensor data measured by the measurement device. The walking waveform processing unit extracts a walking waveform for one gait cycle from sensor data measured according to the movement of the foot. The walking waveform processing unit normalizes the extracted walking waveform. Using the normalized walking waveform, the feature amount construction unit constructs a feature amount related to a sign variable used for estimating a knee pain risk indicating a risk of having a medical examination due to knee pain in the future. The sign variable estimation unit estimates at least one sign variable by inputting the feature amount to a sign variable estimation model that outputs the sign variable used for the estimation of the knee pain risk according to the input of the feature amount. The sign variable estimation unit performs the principal component analysis on the estimated at least one sign variable to generate a principal component vector. The knee pain risk estimation unit estimates the knee pain risk index by inputting the principal component vector to an index estimation model that outputs the knee pain risk index according to the input of the principal component vector. The knee pain risk estimation unit generates knee pain risk information regarding a knee pain risk using the estimated knee pain risk index. The output unit outputs the generated knee pain risk information.

The knee pain risk estimation device of the present example embodiment constructs a feature amount related to a sign variable to be used for estimation of a knee pain risk indicating a risk of having a medical examination due to knee pain in the future based on sensor data measured by the measurement device. The knee pain risk estimation device according to the present example embodiment performs the principal component analysis on the sign variable output from the sign variable estimation model in accordance with the input of the constructed feature amount. The knee pain risk estimation device according to the present example embodiment generates the knee pain risk information regarding the knee pain risk output from the index estimation model according to the input of the principal component vector generated by the principal component analysis. Therefore, according to the present example embodiment, the knee pain risk that may occur in the future can be estimated.

In daily walking, the knee has an important function. Daily knee pain affects the quality of life (QoL) of daily living. For example, when arthritis or the like occurs due to knee osteoarthritis, pain occurs in the knee on a daily basis. Early detection and prevention are important for knee pain. However, at present, for early detection and prevention, measurement using a specialized device and diagnosis by an expert are necessary. Therefore, it is difficult to detect and prevent knee pain at an early stage in daily life. According to the present example embodiment, the knee pain risk can be estimated while living a daily life without using a specialized device.

In one aspect of the present example embodiment, the index estimation model outputs a knee pain risk index indicating a risk of having a medical examination due to knee pain after five years according to the input of the principal component vector. According to the present aspect, it is possible to generate knee pain risk information related to a knee pain risk index indicating a risk of having a medical examination due to knee pain after five years.

In one aspect of the present example embodiment, the index estimation model is a model generated by learning with the principal component vector as an explanatory variable and the knee pain risk index indicating the possibility of having a medical examination due to knee pain in the future as an objective variable. According to the present aspect, the knee pain risk index can be estimated using the index estimation model in which the principal component vector generated by performing the principal component analysis on at least one sign variable and the knee pain risk index are learned.

In one aspect of the present example embodiment, the knee pain risk estimation unit generates knee pain risk information indicating a determination result according to the value of the knee pain risk index output from the index estimation model. According to the present aspect, the knee pain risk information can be generated according to the value of the knee pain risk index.

In one aspect of the present example embodiment, the feature amount construction unit constructs, as a sign variable related to the knee pain risk, a feature amount to be used for estimation of a plurality of sign variables related to the knee joint bending angle and a plurality of sign variables related to the cost indicating the smoothness of the knee movement. The sign variable estimation model includes a first model group and a second model group. The first model group includes a plurality of models for estimating each of a plurality of sign variables related to the knee joint bending angle. The second model group includes a plurality of models for estimating each of a plurality of sign variables related to the cost indicating the smoothness of the knee movement. The sign variable estimation unit inputs a feature amount to be used for estimation of a plurality of sign variables related to the knee joint bending angle to each of the plurality of models included in the first model group, and estimates the plurality of sign variables related to the knee joint bending angle. The sign variable estimation unit inputs, to each of the plurality of models included in the second model group, a feature amount to be used for estimation of the plurality of sign variables related to the cost indicating the smoothness of the knee motion, and estimates the plurality of sign variables related to the cost indicating the smoothness of the knee motion. According to the present aspect, the knee pain risk that can occur in the future can be estimated using the plurality of sign variables related to the knee joint bending angle and the plurality of sign variables related to the cost indicating the smoothness of the knee movement.

In one aspect of the present example embodiment, the first model group includes a plurality of models for estimating each of a plurality of sign variables related to a knee joint bending angle associated with two peaks appearing in time-series data of a knee joint bending angle of one gait cycle. The first model group includes a plurality of models for estimating each of a plurality of sign variables regarding the knee joint bending angle including a temporal relationship between a timing of a peak appearing in the swing phase and a timing of the toe off among two peaks appearing in the time-series data of the knee joint bending angle of one gait cycle. The second model group includes a plurality of models for estimating the sign variable related to the cost indicating the smoothness of the knee motion for each of the plurality of sections included in the stance phase. According to the present aspect, the knee pain risk that can occur in the future can be estimated using the plurality of sign variables related to the knee joint bending angle and the plurality of sign variables related to the cost indicating the smoothness of the knee movement.

In one aspect of the present example embodiment, the knee pain risk estimation device displays knee pain risk information estimated for the user on a screen of a terminal device browsable by the user. According to the present aspect, by displaying the knee pain risk information regarding the user on the screen of the terminal device, the information regarding the knee pain risk can be provided to the user.

### Second Example Embodiment

Next, a knee pain risk estimation device according to a second example embodiment will be described with reference to the drawings. The knee pain risk estimation device of the present example embodiment has a simplified configuration of the knee pain risk estimation device of the first example embodiment.

### Configuration

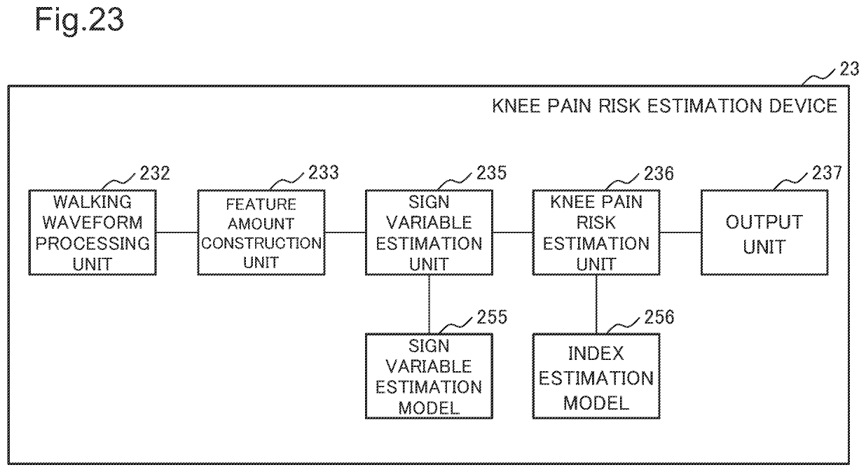

FIG. 23 is a block diagram illustrating an example of a configuration of a knee pain risk estimation device 23 according to the present example embodiment. The knee pain risk estimation device 23 includes a walking waveform processing unit 232, a feature amount construction unit 233, a sign variable estimation unit 235, a knee pain risk estimation unit 236, and an output unit 237.

The walking waveform processing unit 232 extracts a walking waveform for one gait cycle from sensor data measured according to the movement of the foot. The walking waveform processing unit 232 normalizes the extracted walking waveform. Using the normalized walking waveform, the feature amount construction unit 233 constructs a feature amount related to a sign variable used for estimating a knee pain risk indicating a risk of having a medical examination due to knee pain in the future. The sign variable estimation unit 235 estimates at least one sign variable by inputting the feature amount to a sign variable estimation model 255 that outputs the sign variable used for the estimation of the knee pain risk according to the input of the feature amount. The sign variable estimation unit 235 performs the principal component analysis on the estimated at least one sign variable to generate a principal component vector. The knee pain risk estimation unit 236 estimates the knee pain risk index by inputting the principal component vector to an index estimation model 256 that outputs the knee pain risk index according to the input of the principal component vector. The knee pain risk estimation unit 236 generates knee pain risk information regarding a knee pain risk using the estimated knee pain risk index. The output unit 237 outputs the generated knee pain risk information.

### Operation

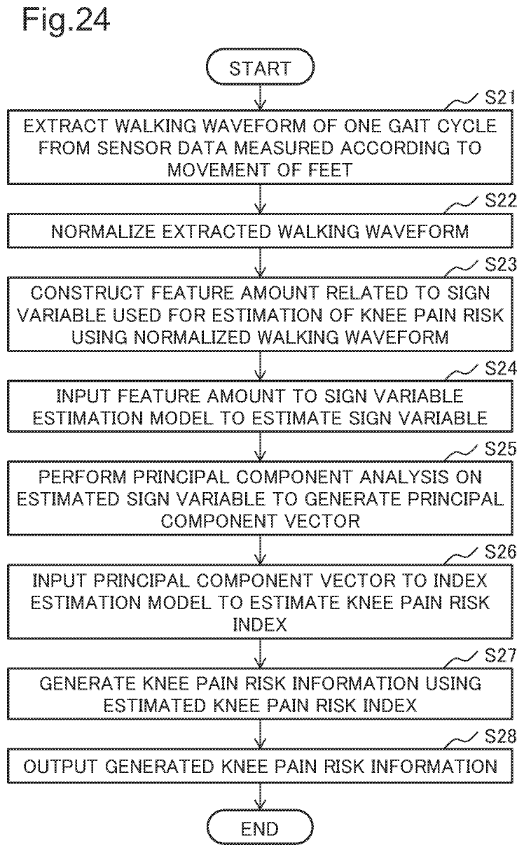

FIG. 24 is a flowchart for explaining an example of the operation of the knee pain risk estimation device 23 according to the present example embodiment. In the processing along the flowchart of FIG. 24, the components of the knee pain risk estimation device 23 will be described as an operation subject.

In FIG. 24, first, the walking waveform processing unit 232 extracts a walking waveform for one gait cycle from sensor data measured according to the movement of the foot (step S21).

Next, the walking waveform processing unit 232 normalizes the extracted walking waveform (step S22).

Using the normalized walking waveform, the feature amount construction unit 233 constructs a feature amount related to a sign variable used for estimating a knee pain risk indicating a risk of having a medical examination due to knee pain in the future (step S23).

The sign variable estimation unit 235 estimates at least one sign variable by inputting the feature amount to a sign variable estimation model 255 that outputs the sign variable used for the estimation of the knee pain risk according to the input of the feature amount (step S24).

The sign variable estimation unit 235 performs the principal component analysis on the estimated at least one sign variable to generate a principal component vector (step S25).

The knee pain risk estimation unit 236 estimates the knee pain risk index by inputting the principal component vector to an index estimation model 256 that outputs the knee pain risk index according to the input of the principal component vector (step S26).

The knee pain risk estimation unit 236 generates knee pain risk information regarding a knee pain risk using the estimated knee pain risk index (step S27).

The output unit 237 outputs the generated knee pain risk information (step S28).

The knee pain risk estimation device of the present example embodiment constructs a feature amount related to a sign variable to be used for estimation of a knee pain risk indicating a risk of having a medical examination due to knee pain in the future based on sensor data measured according to the movement of the foot. The knee pain risk estimation device according to the present example embodiment performs the principal component analysis on the sign variable output from the sign variable estimation model in accordance with the input of the constructed feature amount. The knee pain risk estimation device according to the present example embodiment generates the knee pain risk information regarding the knee pain risk output from the index estimation model according to the input of the principal component vector generated by the principal component analysis. Therefore, according to the present example embodiment, the knee pain risk that may occur in the future can be estimated.

### Hardware

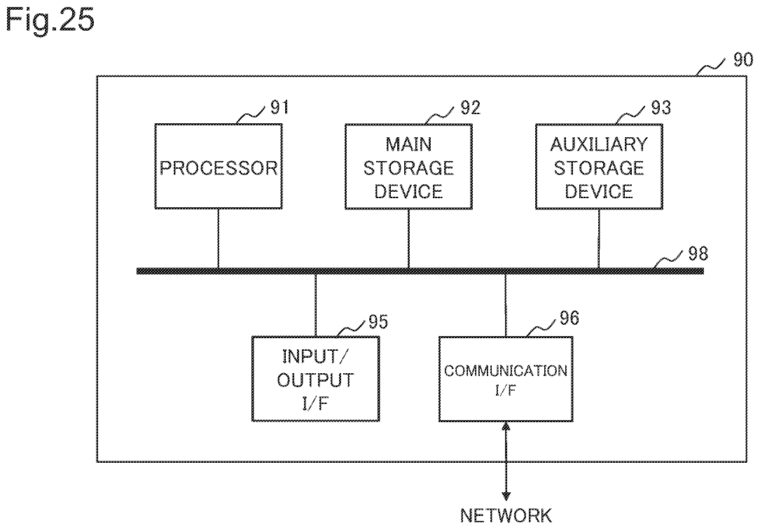

Next, a hardware configuration for executing control and processing according to each example embodiment of the present disclosure will be described with reference to the drawings. Here, an example of such a hardware configuration is an information processing device 90 (computer) in FIG. 25. The information processing device 90 in FIG. 25 is a configuration example for executing the control and processing of each example embodiment, and does not limit the scope of the present disclosure.

As illustrated in FIG. 25, the information processing device 90 includes a processor 91, a main storage device 92, an auxiliary storage device 93, an input/output interface 95, and a communication interface 96. In FIG. 25, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the control and processing of each example embodiment. The processor 91 executes the program developed in the main storage device 92. The processor 91 executes the control and processing according to each example embodiment by executing the program.

The main storage device 92 has an area in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magneto resistive random access memory (MRAM) may be configured and added as the main storage device 92.

The auxiliary storage device 93 stores various data such as programs. The auxiliary storage device 93 is implemented by a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet based on a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When a touch panel is used as the input device, a screen having a touch panel function serves as an interface. The processor 91 and the input device are connected via the input/output interface 95.

The information processing device 90 may be provided with a display device for displaying information. In a case where a display device is provided, the information processing device 90 may include a display control device (not illustrated) for controlling display of the display device. The display device may be connected to the information processing device 90 via the input/output interface 95.

The information processing device 90 may be provided with a drive device. The drive device mediates reading of data and a program stored in a recording medium and writing of a processing result of the information processing device 90 to the recording medium between the processor 91 and the recording medium (program recording medium). The information processing device 90 and the drive device are connected via an input/output interface 95.

The above is an example of the hardware configuration for enabling the control and processing according to each example embodiment of the present disclosure. The hardware configuration of FIG. 25 is an example of a hardware configuration for executing the control and processing of each example embodiment, and does not limit the scope of the present disclosure. A program for causing a computer to execute the control and processing according to each example embodiment is also included in the scope of the present disclosure.

Further, a program recording medium in which the program according to each example embodiment is recorded is also included in the scope of the present disclosure. The recording medium can be achieved by, for example, an optical recording medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium is associated to a program recording medium.

The components of each example embodiment may be made in any combination.

The components of each example embodiment may be implemented by software. The components of each example embodiment may be implemented by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A knee pain risk estimation device comprising:
a memory storing instructions, and
a processor connected to the memory and configured to execute the instructions to:
extract a walking waveform for one gait cycle from sensor data measured according to a movement of a foot and normalizes the extracted walking waveform;
construct, by using the normalized walking waveform, a feature amount related to a sign variable to be used for estimation of a knee pain risk indicating a risk of having a medical examination in a future due to knee pain;
input the feature amount to a sign variable estimation model that outputs a sign variable to be used for estimation of the knee pain risk according to input of the feature amount to estimate at least one of the sign variables;
perform a principal component analysis on the estimated at least one of the sign variables to generate a principal component vector;
estimate a knee pain risk index by inputting the principal component vector to an index estimation model that outputs a knee pain risk index according to an input of the principal component vector;
generate knee pain risk information regarding the knee pain risk by using the estimated knee pain risk index; and
output the generated knee pain risk information.

2. The knee pain risk estimation device according to claim 1, wherein
the index estimation model is configured to output the knee pain risk index indicating a risk of having a medical examination due to knee pain after five years is output in response to an input of the principal component vector.

3. The knee pain risk estimation device according to claim 1, wherein
the index estimation model is a model that is generated by machine learning with the principal component vector as an explanatory variable and the knee pain risk index indicating a possibility of having a medical examination due to knee pain in a future as an objective variable.

4. The knee pain risk estimation device according to claim 1, wherein
the processor is configured to execute the instructions to generate the knee pain risk information indicating a determination result according to a value of the knee pain risk index output from the index estimation model.

5. The knee pain risk estimation device according to claim 1, wherein
the sign variable estimation model includes
a first model group including a plurality of models for estimating each of a plurality of the sign variables related to a knee joint bending angle, and
a second model group including a plurality of models for estimating each of a plurality of the sign variables related to a cost indicating smoothness of a movement of a knee, and
the processor is configured to execute the instructions to
construct the feature amount to be used for estimation of a plurality of the sign variables related to a knee joint bending angle and a plurality of the sign variables related to a cost indicating smoothness of a movement of a knee as the sign variables related to a knee pain risk,
input, to each of a plurality of models included in the first model group, the feature amount to be used for estimation of a plurality of the sign variables related to a knee joint bending angle to estimate a plurality of the sign variables related to a knee joint bending angle, and
input, to each of a plurality of models included in the second model group, the feature amount to be used for estimation of a plurality of the sign variables related to a cost indicating smoothness of a movement of a knee to estimate a plurality of the sign variables related to a cost indicating smoothness of a movement of a knee.

6. The knee pain risk estimation device according to claim 5, wherein
the first model group includes
a plurality of models for estimating each of a plurality of the sign variables relating to a knee joint bending angle associated with two peaks appearing in time-series data of a knee joint bending angle of one gait cycle, and
a plurality of models for estimating each of a plurality of the sign variables related to a knee joint bending angle including a temporal relationship between a timing of a peak appearing in a swing phase and a timing of a toe off among two peaks appearing in time-series data of a knee joint bending angle of one gait cycle, and
the second model group includes
a plurality of models for estimating the sign variable related to the cost indicating smoothness of the movement of a knee for each of a plurality of sections included in a stance phase.

7. The knee pain risk estimation device according to claim 1, wherein
the knee pain risk information is recommendation information optimized according to the knee pain risk.

8. A physical condition estimation system comprising:
a knee pain risk estimation device according to claim 1; and
a measurement device that is installed on a footwear of a user who is an estimation target of the knee pain risk information, measures a spatial acceleration and a spatial angular velocity, generates sensor data according to walking using the measured spatial acceleration and the measured spatial angular velocity, and transmits the generated sensor data to the knee pain risk estimation device.

9. The physical condition estimation system according to claim 7, wherein
the processor of the knee pain risk estimation device is configured to execute the instructions to display the knee pain risk information estimated for the user on a screen of a terminal device browsable by the user.

10. A knee pain risk estimation method causing a computer to execute:
extracting a walking waveform for one gait cycle from sensor data measured according to a movement of a foot;
normalizing the extracted walking waveform;
constructing, using the normalized walking waveform, a feature amount related to a sign variable to be used for estimation of a knee pain risk;
estimating at least one of the sign variables by inputting the feature amount to a sign variable estimation model that outputs a sign variable to be used for estimation of the knee pain risk according to an input of the feature amount;
generating a principal component vector by performing principal component analysis on the estimated at least one sign variable;
estimating a knee pain risk index by inputting the principal component vector to an index estimation model that outputs a knee pain risk index according to an input of the principal component vector;
generating knee pain risk information regarding the knee pain risk using the estimated knee pain risk index; and
outputting the generated knee pain risk information.

11. A non-transitory recording medium having recorded a program causing a computer to execute:
extracting a walking waveform for one gait cycle from sensor data measured according to a movement of a foot;
normalizing the extracted walking waveform;
constructing, using the normalized walking waveform, a feature amount related to a sign variable to be used for estimation of a knee pain risk;
estimating at least one of the sign variables by inputting the feature amount to a sign variable estimation model that outputs a sign variable to be used for estimation of the knee pain risk according to an input of the feature amount;
generating a principal component vector by performing principal component analysis on the estimated at least one sign variable;
estimating a knee pain risk index by inputting the principal component vector to an index estimation model that outputs a knee pain risk index according to an input of the principal component vector;
generating knee pain risk information regarding the knee pain risk using the estimated knee pain risk index; and
outputting the generated knee pain risk information.
